(19) United States
(12) Patent Application Publication
(10) Pub. No.: US 2005/0003022 A1
(43) Pub. Date: Jan. 6, 2005

(54) Broad-spectrum antiviral pharmaceutical composition and manufacturing method thereof

(76) Inventors: Geng-Xin Zhu, Nanjing City (CN); Yi Zeng, Nanjing City (CN); Ze-Lin Li, Nanjing City (CN)

Correspondence Address: Raymond Y. Chan;Suite 128, 108 N. Ynez Ave., Monterey Park, CA, 91754, US

(21) Appl. No.: 10/701,777

(22) Filed: Nov. 4, 2003

(30) Foreign Application Priority Data
Mar. 27, 2002 (CN) 02108282.0

(51) Int. Cl. A61K 35/78

(52) U.S. Cl. 424/725

(57) ABSTRACT

A broad-spectrum antiviral pharmaceutical composition includes total organic and phenolic acid glycosides and/or their condensate compounds and/or salts of total organic and phenolic acid glycosides extracted from a Stellaria plant by a method including the steps of cleaning, powdering and extracting a Stellaria plant to form a water extraction, producing a liquid from the water extraction, absorbing the total organic and phenolic acid glycosides and/or their condensate compounds and/or the salts of total organic and phenolic acid glycosides of the liquid on a resin column by passing the liquid through the resin column, cleaning the resin column by water, washing the resin column by adding an agent to the resin column to obtain and collect a brownish or dark red liquid from the resin column, and collecting the ethanol from the brownish or dark red liquid.

### BACKGROUND OF THE PRESENT INVENTION

1. Field of Invention

The present invention relates to manufacturing method and use of a broad-spectrum antiviral pharmaceutical composition, and more particularly to a broad spectrum antiviral Chinese herb composition and the manufacturing method and use of such broad spectrum antiviral Chinese herb composition.

2. Description of Related Arts

For years, the field of antiviral therapy has sought effective drugs that are capable of treating a wide variety of viral illness. However, until now, there are no drugs available for treating viral infections, particularly, mixed viral infections caused by various viruses or variable viruses. Therefore, how to overcome such long time perplexing problem has been an eminent objective to the international medical community.

Some illnesses caused by viruses are practically lethal, such as HIV, EB virus, rabies virus group, and so on. Meanwhile, other illness, like Hepatitis A, Hepatitis B as well as other Hepatitis infected by 5 kinds of hepatitis viruses, myocarditis caused by Echo virus, Influenza virus could lead patients to lose working ability, temporarily or even permanently. What is more, some viruses are directly responsible initiating cancers.

In short, the illnesses caused by viruses are huge threaten to human being. By far, it has been long-time expectation among international medical community to find some effective broad spectrum antiviral drugs that could inhibit DNA group viruses and RNA group viruses simultaneously, and at the same time, be proven nontoxic. This is due to the fact that only this kind of antiviral drug, which not only could inhibit DNA group virus and RNA group virus as well as its variations but also could be proven secure, will be able to effectively treat illnesses caused by mutable viruses, multi-viruses and mixed infections. In other words, if any drug could inhibit any kind of viruses as well as viral variations, it would be proven effective to treat illness caused by a variety of viruses and mutable viruses.

Commonly, cold could be divided into two categories: common cold caused by bacteria and influenza caused by viruses. Here, Influenza patients covered 75% of total cold cases. Generally, common cold could be treated by antibiotic. However, there weren't any effective drugs for treating influenza caused by viruses until now. All influenza drugs or medicines available on the market today don't provide clear therapeutic function and convincing curative effect. There is no influenza medicines could gain overwhelming recognition.

Treating influenza is challenging, because viruses that could cause influenza are so complicated and from different species. There are 4 types, and more than forty sorts of viruses existed, such as influenza viruses A, B or C, Parainfluenza virus 1 (sendai)-4, and respiratory syncytial virus group of RNA viruses, or adenovirus h1-h34 of DNA viruses. In addition, some of these viruses are still mutable and creating a lot of hypotype viruses from time to time. Since there are too much influenza viruses and non-ceased mutability of viruses, it is impossible to use vaccines to treat influenza. This is due to the fact that most vaccines are exclusively applicable. One kind of vaccine is only shown effective to the virus that it shared the same origin, not for other viruses, specifically for those mutable viruses. Furthermore, vaccines are adapted to precaution only not for treatment.

Sequentially, it is necessary to develop effective therapeutic and clinic broad spectrum antiviral drugs to inhibit DNA and RNA influenza viruses so as to treat the world famous medical problem: Influenza. WHO once expected China could make some contribution to this long term perplexing medical problem. Unfortunately, there are no researches or developments proven effective and efficient until recently.

According to the survey of China, around 4%-37% of Chinese population would have caught influenza annually. Even by a conservative estimation, at least several millions of people would have been caught by influenza annually. Influenza viruses not only deteriorate patient's state of illness if the victim is sick, but also cause accompanying infections frequently, which might lead to rheumatosis, pneumonia, viral myocarditis, and to a worse extent, death. So influenza causes huge influential and damage to our lives.

Accordingly, another American annually influenza report of 1987 indicated that during the first influenza epidemic week in North America 1987, mortality rate around 122 cities in U.S.A. exceed 8% (average rate is 7.2%). And research staffs from University of Wisconsin, in their recent research, found that once particular gene of influenza virus, also known as PB2, varied the toxicity of influenza viruses would increase significantly so as to cause some lethal consequence. Moreover, influenza viruses have been varying constantly, and been able to develop into human being killer at any time. This is a very serious problem.

Until now, there are no antiviral medicine around the world could inhibit so many influenza viruses simultaneously without side effects or toxicity. Moreover, there are no effective influenza vaccines available on the market. So it is said that influenza could be cured automatically within one week no matter whether the patients have been administrated with medicine or not.

Nowadays, some medicines around the world are claimed to be effective to treat influenza. However, none of those medicines can win overall recognition among international medical community.

For instance, Chinese herbs used in clinic application such as Shuang Huang Lian Oral Liquor (SHLOL) et al are failure giving any clear information about how long is the median day of curing process, or how long the state of illness could be reduced and to what extent the influenza symptoms could be alleviated. If an influenza patient took gankang kind of medicine on the fifth day of influenza, he or she would not tell whether the medicine was effective, because it is commonsense that influenza patient will be fully recovered in a week.

In fact, most of medicines proven to be effective in vitro are failure to show expected effectiveness after oral administration as well as liver metabolisms, so as not to treat influenza efficiently. Since all influenza drugs available on current market could not clearly point out the curing period, the international medical community refused giving full recognition for those drugs which claimed efficient in treating influenza. In brief, there is no influenza drug available now won overall acclaim.

For example, taken Shuang Huang Lian Oral Liquor (SHLOL) has been proven for effectively inhibiting influenza viruses and respiratory syncytial virus group during in vitro experiments. However, there is no evidence that it can show same effectiveness after liver metabolism. Meanwhile, it doesn't show any effects for inhibiting parainfluenza viruses, and particularly, adenovirus that can lead to viral pneumonia directly.

Another example, the essential ingredient of “Gankang” is amantadine hydrochloride. It has been used clinically for a long while and its main function is to prevent virus from penetrating host cells, and to thwart viruses' shelling procedure, so as to impact virus' replication. Amantadine hydrochloride has been used to treat Asia-A Virus. If used in time, amantadine hydrochloride can alleviate the influenza symptoms, and specifically, can reduce fever significantly, so as to shorten the illness period as well as reduce accompanying illness. Nevertheless, there are no reports that amantadine hydrochloride can inhibit influenza B or C viruses, parainfluenza viruses, respiratory syncytial viruses, adenoviruses and other viruses caused infections.

About Qilikang, it is reported that some patients, after using it, showed side effect symptoms such as cutis etching all over the body, erythematous eruption, even to the extent, skin measles all over the body. They went to emergency department of hospital and had been prescribed “saigengding and calcium gluconate to treat the eruption. So Qilikang probably have some allergy side effects.

Another widely used old medicine is morpholine biguanidine hydrochloride. It have been said that it is effective in treating influenza viruses such as RNA group and DNA group viruses. However, it is rarely used in clinical practice due to unstable effectiveness.

Ribavirin is a broad spectrum antiviral drug which is shown obvious effectiveness in treating a variety of DNA group and RNA group viruses. Its mechanism is to inhibit viruses from synthesizing nucleic acid so as to impeding virus' replication. Due to its viral inhibiting characteristic, it has been widely used in clinical practice. However, it is failure to show instant effectiveness towards influenza. I.E., it can not shorten the one-week influenza illness period dramatically. Meanwhile, it has some toxic or side effects such as adverse gastrointestinal reaction, anaemia, liver damage, toxicity of pregnant fetal, and respiratory difficulty of infant and asthma patients, and worse, even the to extent death if stop using it.

Influenza vaccines are contributive to prevention of influenza. However, its effectiveness is limited only on precaution, rather for treatment. And due to its strong exclusive characteristic, it is only adaptive for enhancing immunity to some special viruses, instead of treatment after influenza symptom had shown. Another drawback of influenza vaccine is it cost, every injection dosage unit costs 10 dollars. And any health people can not ensure whether he or she will catch influenza within months so as to hardly make the vaccine injecting decision. In other words, the influenza is not very convenient in preventive application. Furthermore, according to an article “wrong direction of health care” of Science Abstract magazine, January 2001 edition, influenza vaccine is actually one sort of virus. It had enormous side effect such as causing senior patient's pathologic immunity reaction so as to damage their immune system as well as initiate cancer indirectly.

According to some Newspaper and Pharmacy medical magazine, the best selling drug for influenza is neuraminidase inhibitors including Zanamivir and Oseltamivir phosphate, registered as Relenza and Tamiflu separately. This kind of influenza drugs has been widely used in Europe, North America and Australia since 1999. Its main advantage is strong inhibiting effectiveness in treating influenza virus A and B.

In addition, according to the report, the median day of Zanamirvir for alleviating influenza symptoms is 6.5 days. Compared with normal cases, it reduces 1.3 day from average influenza case. This is by far the most significant achievement in medical industry to treat influenza. However, inhibiting influenza virus A and B can not satisfy the requests from therapeutic treatment. This kind of drug still has some deficiencies and drawbacks.

1. Its median day of alleviating influenza symptoms is 5.2 days. In comparison with ordinary 6.5 days influenza period, it only reduces 1.3 day. Even though it is a significant achievement, it still needs influenza patient 5 suffering days to be recovered.

2. Neuraminidase inhibitors can inhibit Influenza viruses A and B. However, there are no reports publicized for neuraminidase inhibitors' effects in treating other viruses which can cause respiratory infections, such as influenza virus C, rhinovirus, ECHO virus, parainfluenza virus, respiratory syncytial virus group and DNA group adenoviruses. So its function and practice are within limitation.

3. The reports regarding its effectiveness about headache symptom of influenza are unseen.

4. Neuraminidase inhibitors have some side effects such as nausea, vomiting and hypersensitivity. Particularly, during the first three month of pregnancy, Zanamivir administrating should be forbidden without prescriptions.

5. Zanamivir is indicated for the treatment of infections due to influenza A and B viruses in adults and adolescents (≧12 years).

6. The utilization ratio of oral taken Zanamivir is only 2%, so it is not suitable for oral administration.

7. One package of Zanamivir on the market costs around 25 dollars, Oseltamivir costs around 30 dollars. It is too expensive to be affordable for many developing country customers.

Conclusively, international medical community recognized that neuraminidase inhibitors such as Zanamivir and Oseltamivir have limited effectiveness in treating influenza. However, they are still far from satisfactory solution to influenza.

As far as the antiviral drugs concerned, British company “Burroghs Wellcome” had developed antiviral nucleotide drugs “axiluowei” which is active against simplex herpes and zoster herpes. It had been first unveiled on British market in 1981 and had been approved by FDA in 1982. Chinese medical industry institution had trial-produced this medicine. However, there is no obvious evidence that “axiluowei” are efficient in treating viral illness.

Interferon, developed from modern gene engineering, had been widely used in clinical practice for antiviral purpose. Its therapeutic effect in curing viral hepatitis is successful. And interferon is also adapted for treating herpes illness. Its major drawback is expensive costs and long period of time therapeutic injection. Moreover, interferon is not suitable to all species of viral illness.

Nucleic acid group medicines as well as their cocktail treating method, adapted for treating AIDS, such as Azidothymidine and Zalcitabin, have been proven effective alleviating symptoms distinctly so as to prolong patients' life. This is due to the fact that by using the cocktail treating method, various types of pharmaceutics had been combined and interchanged to treat HIV. Nevertheless, HIV, like influenza viruses, is unsteady virus and fallen into different species. Even under normal circumstance without external factors, it is still susceptible to mutation. There are more than 10 types of HIV discovered all around the world, and at least 8 types of HIV have been identified in China. Nucleic acid medicines could not cure all these types of HIV. Moreover, its clinical practical is restrictedly limited because of considerable side effects of nucleic acid medicines. Another disadvantage factor of Nucleic acid medicine is expensive costs, so ordinary patients would be deemed unaffordable using it in a long period of time.

In brief, all antiviral drugs available on market, proven efficient for some viral illness, are limited to some exclusive applications. And these antiviral drugs either have much toxicity or side effects or have practical limitation. Meanwhile, cost and convenience factor further restricted their application. So the international medical community had been eagerly expected to develop an effective, convenient, and safe broad spectrum antiviral pharmaceutics to treat viral illnesses caused by various and mutable viruses.

### SUMMARY OF THE PRESENT INVENTION

A main object of the present invention is to provide a strong and broad-spectrum antiviral nature remedy from Stelllaria L or Stellaria group of plants, which can inhibit both RNA group viruses and DNA group viruses simultaneously and effectively in low cost, without causing any evident toxicity. As a result, it could fill the gaps of science and technologies about treating viral illness and provides pharmaceutical composition and method based on natural herb.

Another object of the present invention is to provide a broad-spectrum antiviral pharmaceutical composition which is an extract Chinese herb composition, consisting of total (organic) phenolic acid glycosides, salts of total (organic) phenolic acid glycosides and condensate compounds thereof from Stellaria media (L.) or Stellaria L. as well as its glycosides, and their condensate compounds from Stellaria media (L.) Cyr or Stellaria L plants. This composition may further include elements of sulfur, nitrogen, and calcium and so on. The MW 564 component from this composition has been isolated and identified as 6-C-glu, 8-C-ara apigemin glycoside; it is a new compound and called Stellarinol A. In addition, when this composition is heated with dilute inorganic acid, this composition can turn out a red phlobaphene, further illustrating that the composition contains flavanoids constituents and their condensate constituents.

Hereinafter, composition comprising mixtures of the above extracts is called “Total Stellaria Phenolic Acids Glucosides” (TSPAG). According to the articles of American periodical “Chemical Abstracts” from 1920 to 2002, the main chemical constituents of Stellaria L comprise flavone and flavone glycoside, saponin, amidocarbonic acid, chlorogenic, neochlorogenic, ferulic, ethanedioic, caffeic, quinic acid, acyl-galactose lipid, organic alkali, etc. Most constituents mentioned herein are medium polar compound. Those articles did not deal with much about water soluble big polar compound, or introduce intensive research about compound comprising element of sulfur, nitrogen and calcusium.
Another object of the present invention is to provide a broad-spectrum antiviral pharmaceutical composition which is related to big polar compound extracted from Stellaria L, especially relates to water soluble big polar compound comprising nitrogen, calcium and sulfur extracted from Stellaria L.

Another object of the present invention is to provide a broad-spectrum antiviral pharmaceutical composition, which is related to Stellaria L. and Malachium Fries comprising 77 species: Stellaria alsine Grimm, Stellria alsine var. phaeuspetala Hand.- Mazz, Stellaria anhwiensis Migo, Stellaria amblyosepala Schrenk, Stellaria apiculata Wils, 4987Stellaria aquatica(L) Scop, Stellaria arenaria Maxim, Stellaria arisanensis Hayata, Stellaria arisanensis var. leptophylla Hayata, Stellaria borealis Bigel, Stellaria brahypetala Bge, Stellaria bungeana Fenzl, Stellaria cherleriae(Fisch.) Will, Stellaria chinensis Regel, Stellaria cicrantha, Stellaria crispate Wall, Stellaria davidii Hemsl, Stellaria crispate Wall, Stellaria davidii hemsl, Stellaria decumbens Edgew., Stellaria decunbens var. acicularia Edgew.Et Hook.f., Stellaria delavayi Franch, Stellaria dianthifolia Williams, Stellaria dichasioides Williams, Stellaria dichotoma L., Stellaria dichotoma var. lanceolata Bge., Stellaria dichotoma var. stepheniana Willd., Stellaria diffusa Wills., Stellaria discolor Turcz., Stellaria diversiflora Maxim., Stellaria diversiflora var. gymnandra Franch., Stellaria depressa Schnid., Stellaria duthiei Gandoger, Stellaria filicaulis Mak., Stellaria filipes Komar., Stellaria florida Fisch., Stellaria graminea L., Stellaria graminea var. pilosula Maxim., Stellaria graminea var. viridescens Maxim., Stellaria diversiflora var. gymnandra Franch., Stellaria depressa Schnid., Stellaria duthiei Gandoger, Stellaria filicaulis Mak., Stellaria flipes Komar., Stellaria florida Fisch, Stellaria graminea L., Stellaria graminea var. pilosula Maxim., Stellaria graminea var. viridescens Maxim., Stellaria gyantsensis Williams, Stellaria gypsophiloides Fenzl., Stellaria henryi Williams, Stellaria hsinganensis Kitagawa, Stellaria infracta Maxim., Stellaria leptophylla Hance, Stellaria maximowixziana Franch., Stellaria media(L.) Cyr., Stellaria micrantha Hayata, Stellaria mitans Williams, Stellaria neglecta Weihe, Stellaria neo-palustris Kitagawa, Stellaria octandra Fobedim., Stellaria oxycoccoides Komar., Stellaria palustris L., Stellaria patentifolia Kitagawa, Stellaria pseudosaxatilis Hand.-Mass., Stellaria pusilla Schmid., Stellaria radians L., Stellaria reticulivena Hayata, Stellaria rupestris Hemsl, Stellaria saxatilis Buch-Ham, Stellaria saxatilis var. amplexicaulis Hand.-Mazz., Stellaria soongorica Roshev., Stellaria souliei Williams, Stellaria stellato-pilosa Hayata, Stellaria stongylosepala Hand.-Mazz., Stellaria subumbellata Edgew., Stellaria tomentella Ohwi, Stellaria trimorpha Nakai, Stellaria turkestanica Schischk, Stellaria uda Williams, Stellaria umbellate Truez., Stellaria virdiflora Pax et O. Hoffm., Stellaria wushanensis Williams, Stellaria wutaica Hand.-Mazz., Stellaria yunnanensis franch. J and Malachium aquaticum (L.) Fries.
According to Chinese herb pharmacology, Stellaria L has the following characteristics:

(i) Nature of drug: sweet with a little bit salty, flat taste, nonpoisonous.

(ii) Action: activating blood circulation and clearing away blood stasis, mainly for cases of postpartum anemia with dull aching over lower abdomen, insufficient latex, summer heat caused vomiting, periappendicular abscess, gonorrhea, toxic sore, swelling, and beating wound.

(iii) Using direction: for oral use, simmering 50-100 gram, or directly pounding; for external use, pounding and spreading.

The present invention has the following chemical and physical characteristics:

1) On the basis of colors, TSPAG can be further divided into three portions: brownish black portion called TSPAG-A, brown portion called TSPAG-B and yellow portion called TSPAG-C. The antiviral efficacy of TSPAG-A color is the strongest, and the antiviral efficacy of TSPAG-C color is weakest.

2) TSPAG, especially its brownish black color portion, has elements of sulfur, nitrogen, and calcium. (Resin rosin resinous)

3) TSPAG can be absorbed by DEAE cellulose or anion exchange resin. TSPAG is acidic.

4) TSPAG will be changing to dark brown color in case of reacting with ferric trichloride.

The present invention has following advantages effects:

1) TSPAG has shown nontoxic characteristic after in vivo acute poisonous animal experiment. (acute toxicity test)
I. The maximum tolerance dosage of small mice by nasal mucosa administration (MTD) is 2.25 g/kg, with no LD₅₀ discovered.
II. The maximum tolerance dosage of large mice by nasal mucosa administration (MTD) is 1.125 g/kg, with no LD₅₀ discovered.
III. The maximum tolerance dosage of small mice by oral administration n is 11.25 g/kg, with no LD₅₀ discovered.
IV. LD₅₀ of TSPAG according to intravenous administration is 3.406 g/kg, its 95% credibility limitation is from 3.069 g/kg to 3.780 g/kg.

2) The present invention didn't show any irritative effects after large mice in vivo experiment by nasal mucosa administration (MTD).
I. There are no toxic effects shown after experiments using different methods such as maximum concentration, maximum administrative capacity, daily once administration, and one week continuous administration. 24 hours after the experiment, the nasal cavities of mice were dissected. There are no symptoms like swelling, ulcers, and congestion has been identified. The mucous membrane condition has shown no difference in comparison with of normal saline administration.
II. There are no toxic effects shown after experiments using different methods such as maximum concentration, maximum administrative capacity, daily continuous administration, and one week continuous administration. 24 hours after the experiment, the nasal cavities of mice were dissected. There are no symptoms like swelling, ulcers, and congestion has been identified. The mucous membrane condition has shown no difference in comparison with of normal saline administration group.

Above information illustrate that TSPAG is one kind of nature medicine with extreme safety, since there are no obvious toxicity and irritation.

3) The present invention has shown antiviral effectiveness: TSPAG having strong broad spectrum antiviral effects.
I. The developed experiments have shown that TSPAG can effectively inhibit various respiratory system viruses such as influenza viruses, Parainfluenza viruses, rhinovirus, ECHO virus, herpes viruses belonging to RNA group viruses and DNA group adenoviruses. Particularly, TSPAG can strongly inhibit influenza virus, parainfluenzaviruses, and adenoviruses. This fact further supports TSPAG as one kind of broad spectrum antiviral drugs.
II. According to the small mice in vivo experiment for influenza virus test, TSPAG showed strong inhibiting effectiveness towards influenza virus. The small mouse protection ratio (ED50) is 0.51 mg/kg, for the maximum tolerance dosage of small mice by nasal mucosa administration (MTD) is 2250 mg/kg. So TSPAG' treatment index (TI), based on small mice nasal mucosa administration (MTD) is 4412. (TI=MTD/ED50=2250/0.51=4412) The anti-influenza effectiveness of TSPAG has been fully illustrated because 4412 is so high for TI.

Compared with Traditional Chinese medicine Shuang Huang Lian Oral Liquor, ED₅₀ being 866 mg/kg, TSPAG's TI is estimated to be 1700 times better.

Compared with Ribavirin, ED50 being 25 mg/kg, LD50 (small mouse 50% lethal ration) being 2000 mg/kg, TI being estimated 80 (TIRibavirin=LD50/ED50=80), TSPAG's TI is 56 times better.

In short, TSPAG has shown effective inhibiting characteristics as a broad spectrum antiviral drug. Its practical performance is much better than that of Shuang Huang Lian Oral Liquor and Ribavirin.
III. Clinical practice effects:

According to our statistics, the nasal drop dosage of TSPAG, 5 ml solvent with 0.2 g-0.4 g TSPAG, can fully recover influenza patient within 2 days. Its concentration scopes from 40 mg/ml-80 mg/ml, 120 drops/5 ml, and each nostril is used two drops for administrative practice. The actual administration of TSPAG is around 6 mg/person-12 mg/person each time. This nasal dosage can be used 30 times within two days from 7 am to 21 am. The administration interval is one time per hour. Based on clinical practice in last six years, more than 100 patients have been treated by TSPAG. No matter how long the patient had caught the influenza, he or she would be gradually relieved from influenza symptoms as well as fully recovered within two practical days after being administrated TSPAG. In two days, influenza patient could resume normal health condition. For instance, within an hour after taking TSPAG, patients could be relieved from headache, nasal congestion, nasal discharge, tears, as well as overall paining; within 24 to 48 hours, TSPAG could reduce fever efficiently. Furthermore, the earlier influenza patient took TSPAG, the more effectiveness TSPAG would have shown. In our statistics, one patient was fully recovered from his serious symptoms overnight, (his symptoms including headache, overall paining, high fever 39.5 C, tearing, and nasal discharging), after used TSPAG before going sleep. He could go to school and resume his job the second day. These cases illustrated that TSPAG was of extreme effectiveness beyond ordinary imagination.

According to theoretical calculation from virology institution, Chinese academy of preventive medicine, ED50 of TSPAG equals to 50 mg/kg. Divided by surface coefficient index 10, the quotient will be 0.05 mg/kg, then multiply weight of people, 60 kg, the final result is 3 mg/per time/per person. If this figure were doubled, it would be 6 mg/per time/per person. This ultimate result could be matched by dosage unit in clinical practice, 6 mg/per time/per person to 12 mg/per time/per person. So above information provide sounding evidence and support of TSPAG's clinical application.

During the long period of time clinical practice, there is no toxicity or distinct side effects shown among patients who have been administrated TSPAG for treating influenza. As mentioned above, the maximum tolerance dosage of small mice experiment by nasal mucosa administration (MTD) is 2250 mg/kg, no toxic symptoms have been identified. Actually, the actual practical dosage is only ranged from 6 mg/per time/per person to 12 mg/per time/per person, the total dosage in two days therapeutic period is around 0.2 g to 0.4 g. It is impossible to cause any toxic and side effects.
IV. Cellular experiment showed TSPAG has been inhibiting effectiveness towards HIV- 1.
V. Primary stage cellular experiment illustrated TSPAG can inhibit hepatitis virus B and e-antigens.

Conclusively, the present Chinese medicine composition, which comprises total organic phenolic acid/salt of total organic phenolic acid as well as glycoside, extracted from Stellaria L, are illustrated and proven safe and effective.

It will be shown that there are provided compositions and methods which achieve the various objects of the invention, and which are well adapted to meet the conditions of practical use.

Still further objects and advantages will become apparent from a consideration of the description and drawings.

These and other objectives, features, and advantages of the present invention will become apparent from the following detailed description, the accompanying drawings, and the appended claims.

### BRIEF DESCRIPTION OF THE DRAWINGS

FIG. 1 is an ultraviolet scanning diagram for TSPAG, in which greatest adsorption peaks located on UV λ 272 nm, 327 nm.

FIG. 2 is an infrared scanning diagram for TSPAG, in which distinguishbilities happened on IR υₘₐₓ(KBr)cm⁻¹: 3400(hydroxyl peak), 1653(carbonyl), 1625, 1580 and 1508(benzol).

FIG. 3 is an infrared scanning diagram of Stellarinol A, wherein IR υₘₐₓ(KBr)cm⁻¹: 3400(hydroxylpeak), 1653(carbonyl), 1625, 1580 and 1508(benzol)etc.

FIG. 4 shows ion flow of mass spectra molecule from TSPAG-C. It is shown ESI(−) 70V, Max 986: 1129, 960, 708, 561, 447, 365, 337, 283, 255, 141.

FIG. 5 shows MS m/e 563.2(M-1)⁻ of Stellarinol A.

FIG. 6 illustrates mass spectrum 1 of TSPAG-A portion, in which comprises Ion peak ESI(−)70V, Max: 370 m/z: 563, 593, 594, 901 [symbol] 127, 136, 161, 243, 259, 283, 305, 311, 327, 335, 336, 360, 391, 423, 465, 517, 563, 609, 623, 651, 655, 657, 692, 747, 748, 749, 754, 755, 757, 776, 777, 778, 791, 810, 815, 818, 834, 909, 939, 951, 996, 1056, 1134, 1137, 1143, 1197, 1269, 1296, 1348, 1521, 1651, 1671, 1675, 1709, 1730, 1744, 1794, 1818, 1819, 1864, 1989, 2121, 2177, 2326, 2497, 2509, 2668, 2799, 2915 etc.

FIG. 7 illustrates mass spectrum 2 of TSPAG portion, in which TSPAG comprises Ion peak ESI(+)100V, Max: 700 m/z: 587 [symbol] 119, 124, 125, 135, 136, 137, 138, 152, 165, 175, 211, 212, 242, 243, 244, 315, 353, 354, 355, 360, 393, 409, 410, 411, 412, 439, 449, 450, 538, 565, 588, 603, 604, 608, 609, 616, 617, 618, 621, 637, 638, 639, 681, 682, 688, 699, 700, 719, 750, 877, 889, 900, 925, 1124, 1317, 1464, 1528, 1603, 1651, 1753, 1782, 1864, 1927, 1976, 2049, 2115, 2231, 2278, 2293, 2329, 2356, 2361, 2398, 2463, 2489, 2545, 2560, 2594, 2620, 2680, 2923, 2984 etc.

FIG. 8 illustrates mass spectrum 3 of TSPAG-C portion, in which TSPAG comprises Ion peak (M-1) 593.0, 360.2, 417.1, 563.1, 834.3 components.

FIG. 9 illustrates mass spectrum 4 of TSPAG-C portion, in which TSPAG comprises Ion peak (M-1) 755.1, 296.0, 593.0, 623.1, 791.0 components.

FIG. 10 is ¹³C NMR spectra of Stellarinol A(DMSO D-6 )δ: 182.449 (s, 4-C), 164.381 (s, 2-C), 161.508 (s, 7-C), 161.292 (s, 4′-C), 158.538 (s, 5-C), 157.545 (s, 9-C), 129.674(2d, 2′6′-C),121.238 (s, 1′-C), 116.132(2d, 2′,6′-C), 108.251 (s, 6-c),105.106 (s, 8-C), 103.7069(s, 10-C), 102.318(d, 3-C), 79.365(d, -C), 75.254(d, -C), 74.802(d, -C,) 74.678(d, -C), 73.849(d, -C), 71.227(d, -C), 69.218(d, -C), 68.818(d, -C), 68.665(d,-C), 68.510(d, -C), 60.956(d, -C).

FIG. 11 is ¹H NMR spectra of Stellarinol A (DMSO D-6)δ: 13.8008 (1H, S, 5-OH), 10.2670 (1H, s, 7-OH), 9.1599 (1H, s 4′-OH), 8.2734 (2H, br,s, 2′,6′-H), 6.9028(2H, d, j=8.4, 3′,5′-H), 6.8389 (1H, s 3-H),

FIG. 12 is ¹H NMR spectra of Stellarinol A in DMSO D-6+D₂O solvent.

FIG. 13 is a TSPAG's definite HPLC-fingerprinter as the quality standard, in which the testing condition is: C₁₈ column, testing wavelength: 360 nm, 256 attenuation, 0.25 cm/min paper speed; eluting with different concentrated H₂O and MeCN mobile phase; and controlling the peak of Stellarinol A around Rt21 min.

FIG. 14 is a TSPAG's element analytical chart of X ray-fuosrescent splitting luminosity detection further showing substantial elements of sulfur, and calcium so as to support TSPAG's characteristics with sulfur and calcium.

FIG. 15 is a constituents pump off ultraviolet scanning chart of five peaks of TSPAG's HPLC, its scanning area: 190 nm-400 nm; 192 nm peak of five peaks' constiuents are marked as “6”, “17”, “27”, “36” and “43” respectively; 202 nm peak of five peak's constituents are marked as “9”, “18”, “28”, “37”, “44” respectively; 275 nm peak of five peak's constituents are marked as “10”, “19”, “29”, “38”, “45” respectively; 338 nm peak of five peak's constituents are marked as “11”, “20”, “30”, “39”, “46” respectively, in which since the greatest ultraviolet absorbing peak of different peak's constituent are similar, the chromophore group and basic structure of TSPAG's main constituents are almost identical.

FIG. 16 is Report 1 showing the analytical result of nitrogen, carbon, hydrogen elements of brownish black portion of TSPAG, which comprise 8% nitrogen, 47% carbon, 5.5% hydrogen.

FIG. 17 is Report 2 showing the analytical result of X-ray and fluorescent spectroscopic luminosity showing TSPAG comprises sufficient elements of sulfur and calcium.

FIG. 18 is Table 1 showing the experimental result of drug effect based on index such as mice lethal rate, protection rate of animal in vivo experiment.

FIG. 19 is Table 2 showing the 50% effective dose (ED50 ) based on mice protection rate and lives extension rate.

FIG. 20 is Table 3 showing the experimental result of drug effect based on index of pulmo pathologic process of mice.

FIG. 21 is Table 4 which is an attached list of Table 3 in FIG. 27, further illustrating experimental result of drug effect based on index of viral pneumonia.

FIG. 22 is Table 5 showing the result of four times cellular experiment about TSPAG's inhibiting effectiveness towards HIV-1.

FIG. 23 is Table 6 showing TSPAG's clinical effects: with 0.4 g of TSPAG at least could reduce 5 days treatment period.

FIG. 24 is Table 7 showing TSPAG's actual effect for clinical practice in treating simplex herpes virus and zoster herpes virus.

FIG. 25 is Table 8 showing TSPAG's effect in treating parotitis of children.

FIG. 26 is Table 9 showing TSPAG' effect in treating venereal disease venereal wart wherein it is effective.

FIG. 27 is Table 10 showing the antiviral effectiveness of the present invention used as disinfectant for influenza and parotitis prevention.

FIG. 28 is Table 11 showing the embodiment of the present invention used as health care products for influenza and parotitis prevention.

FIG. 29 is Table 12 showing the difference of obtaining rate of various ethanol. concentrations used in the above first preferred embodiment.

FIG. 30 is Table 13 shows the difference of obtaining rate according to different Stellaria L. used in the above first preferred embodiment.

### DETAILED DESCRIPTION OF THE PREFERRED EMBODIMENT

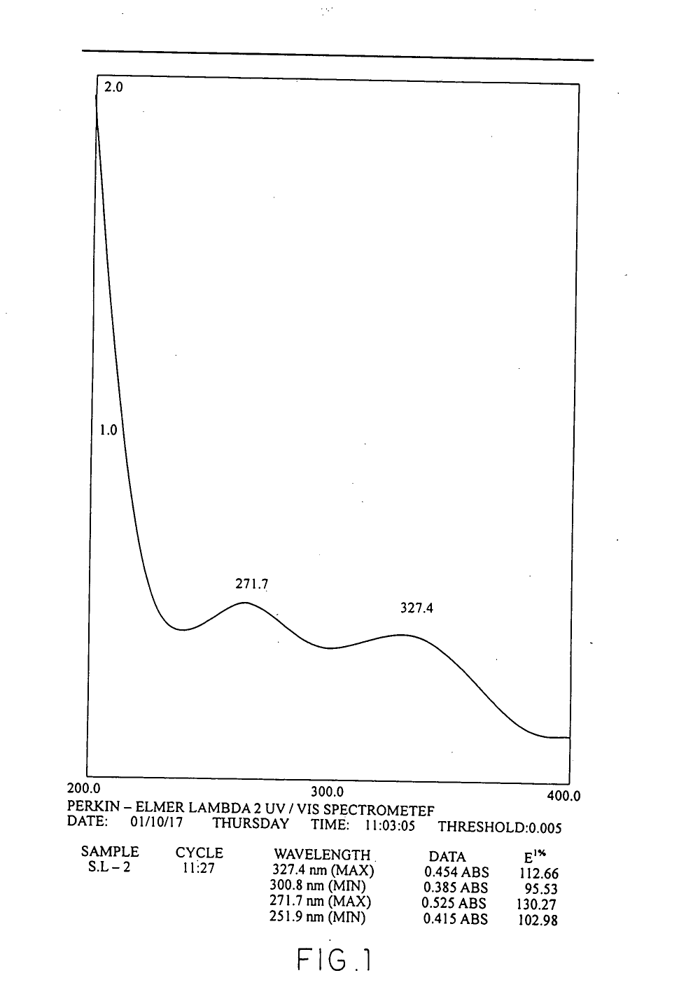

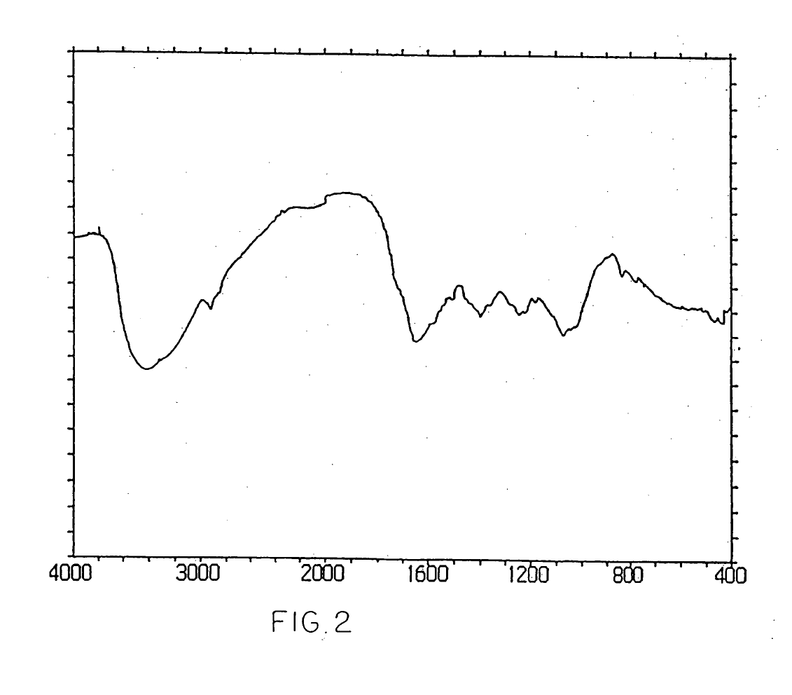

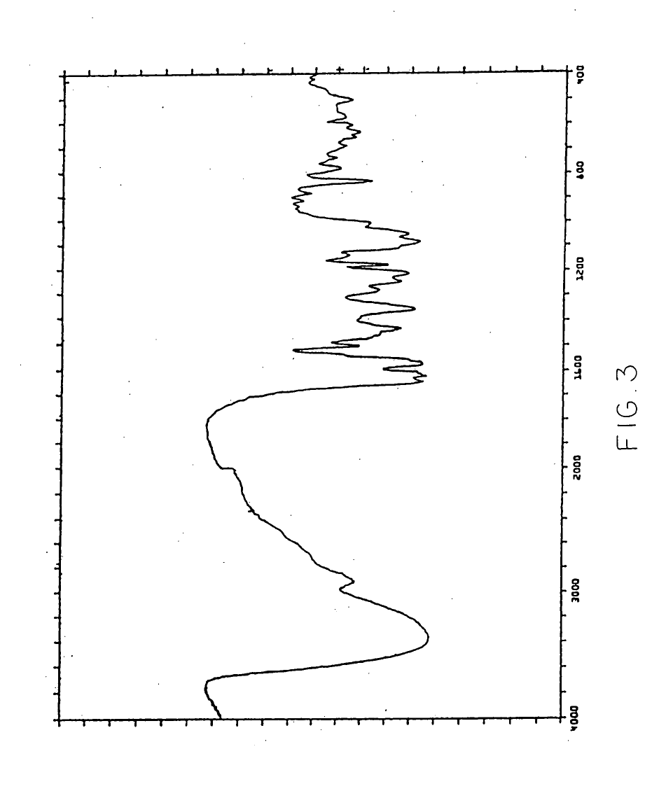

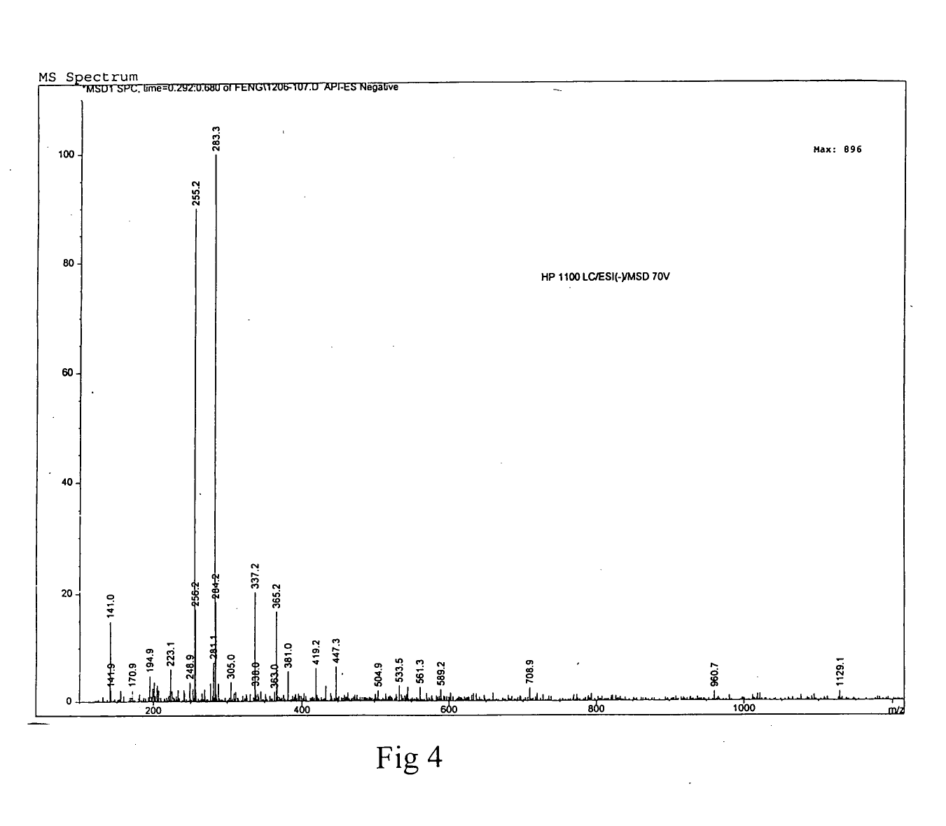

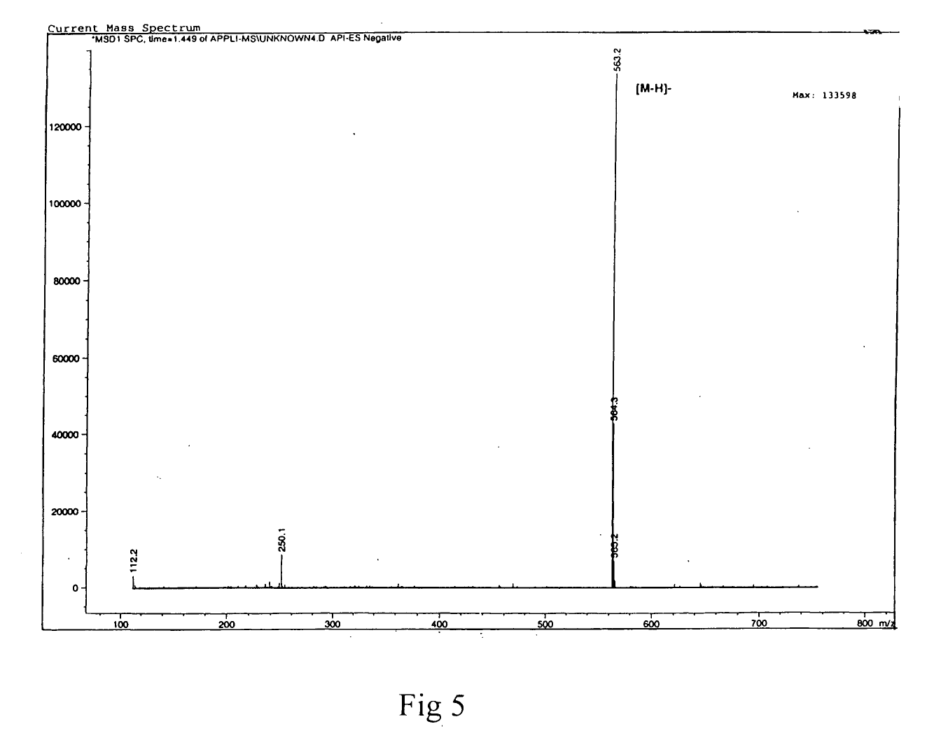

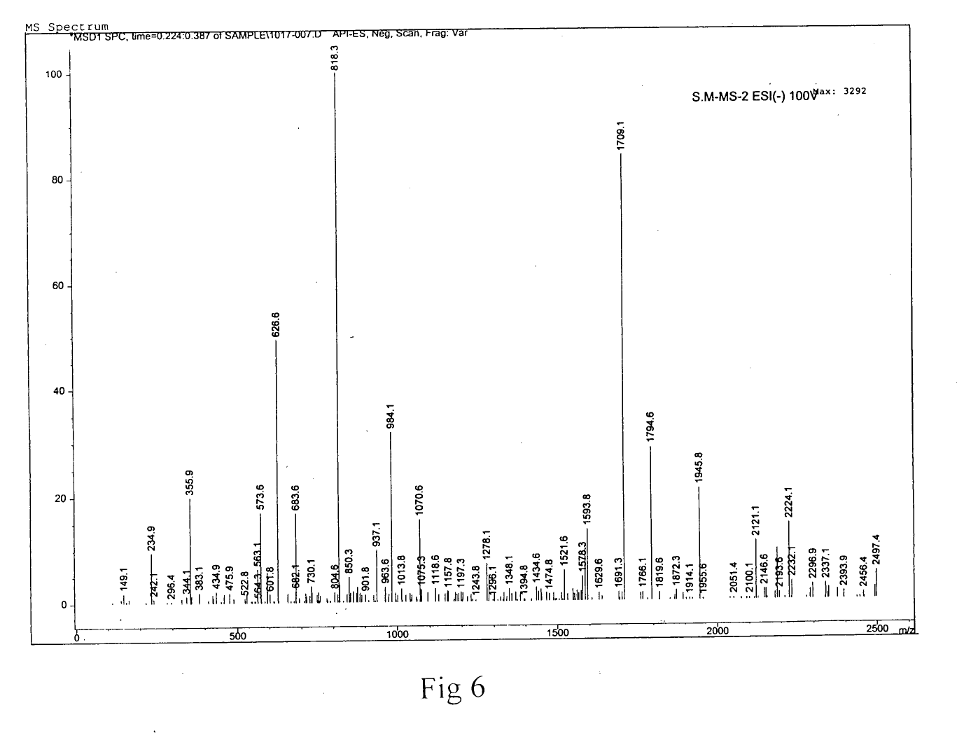

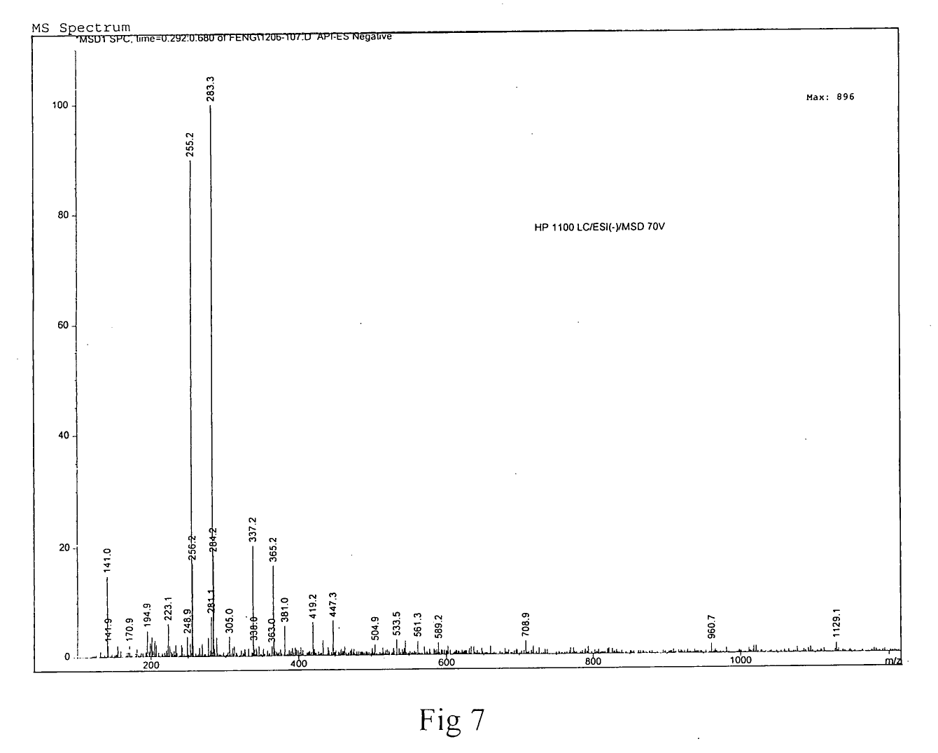

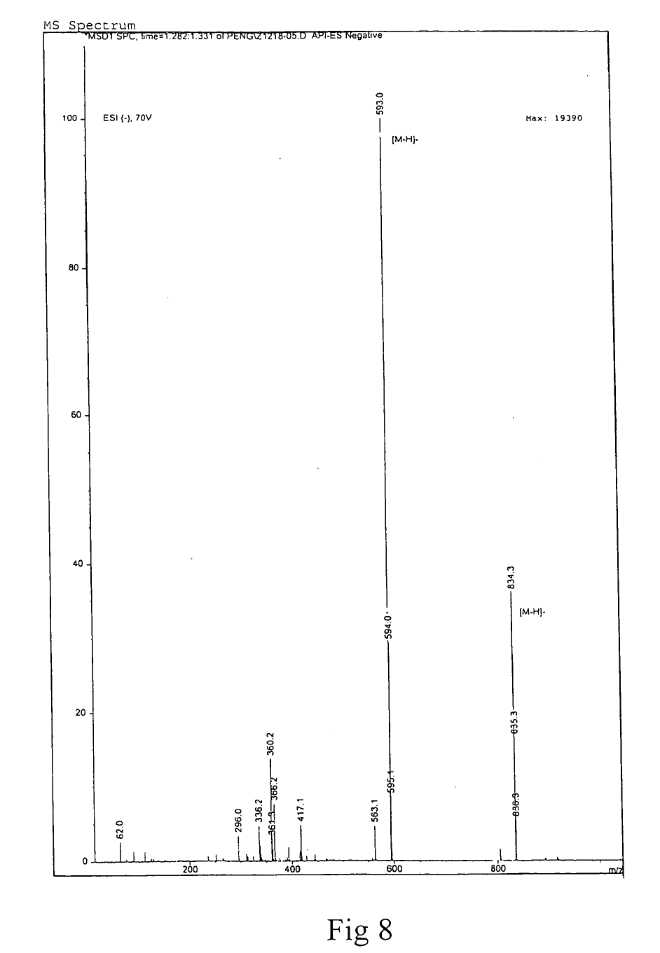

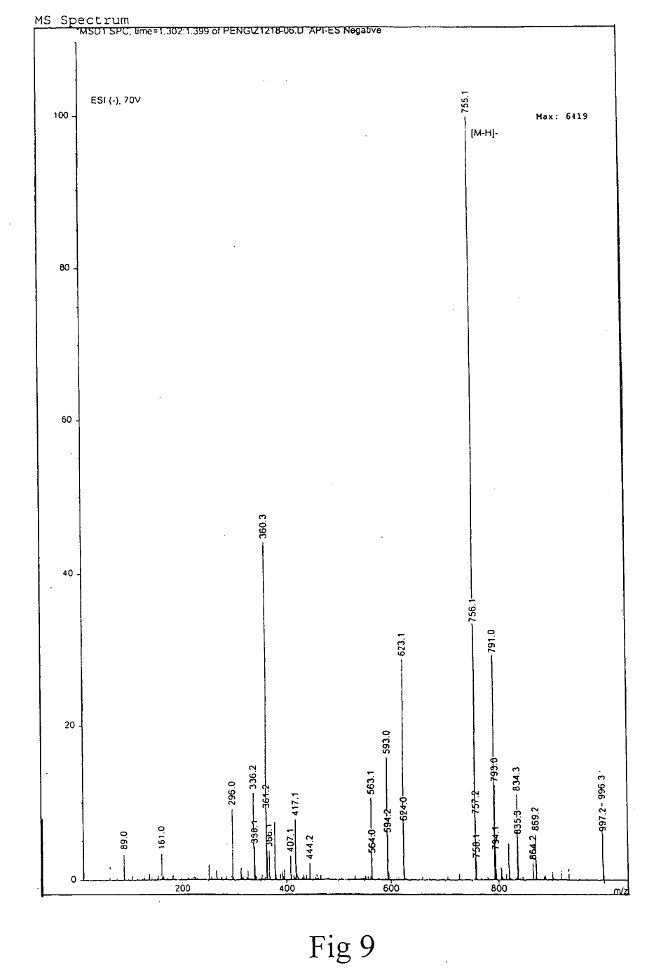

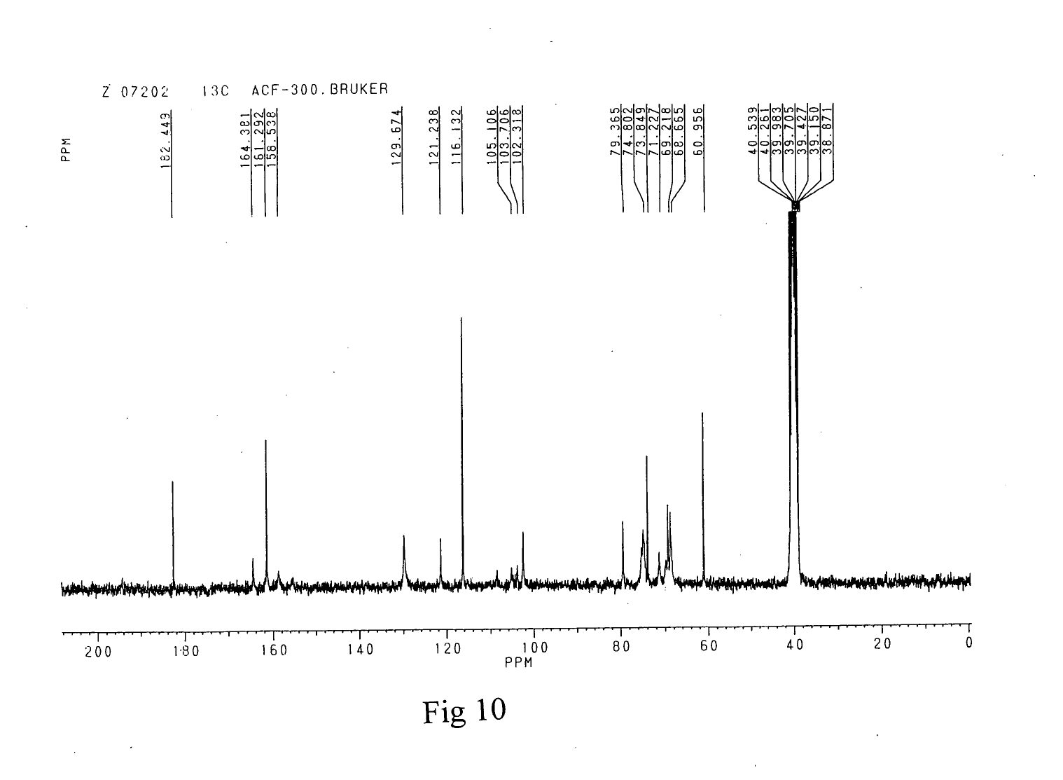

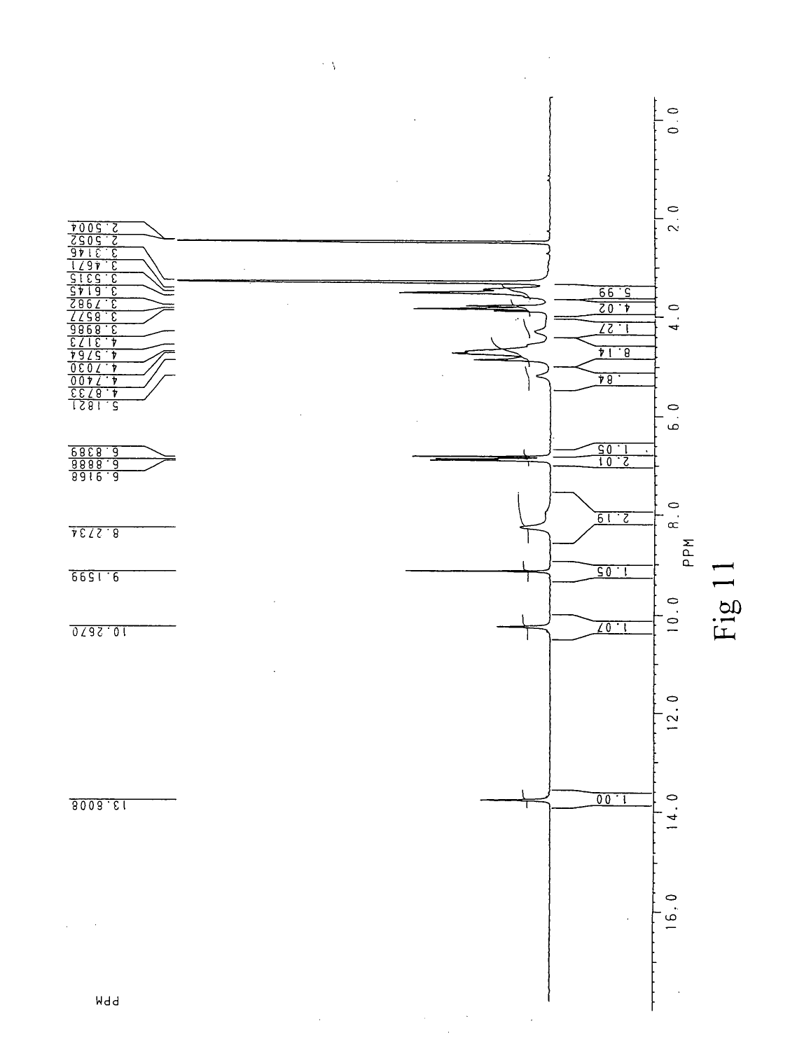

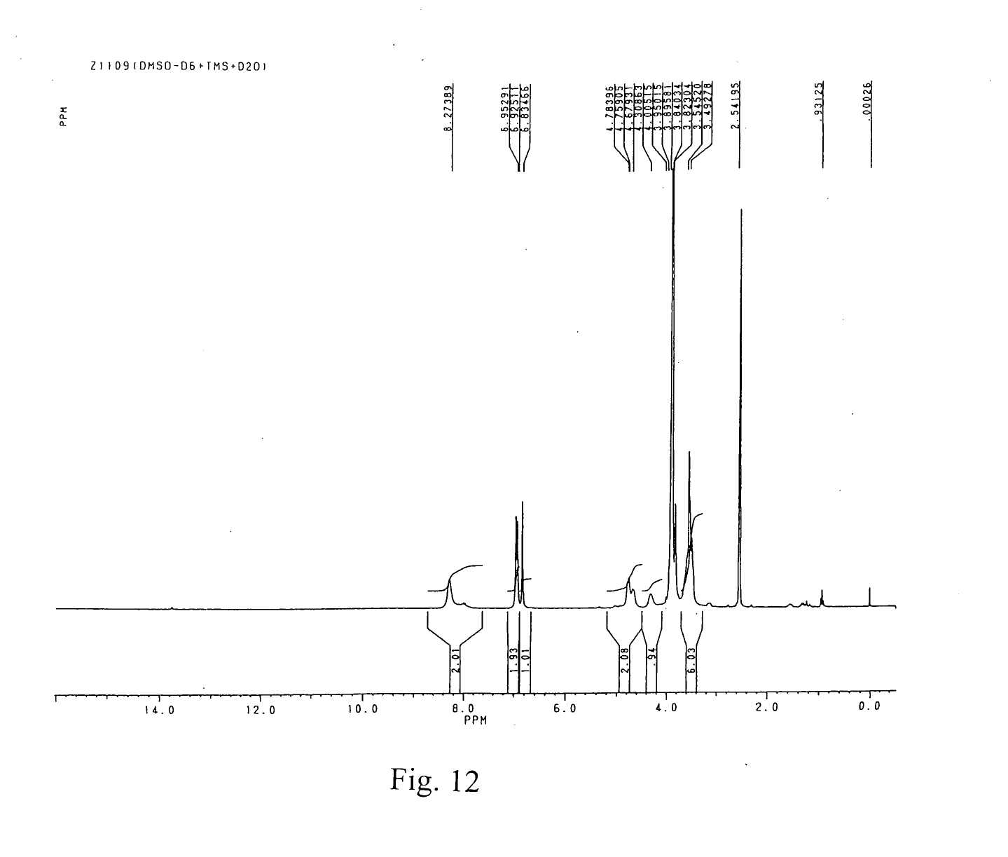

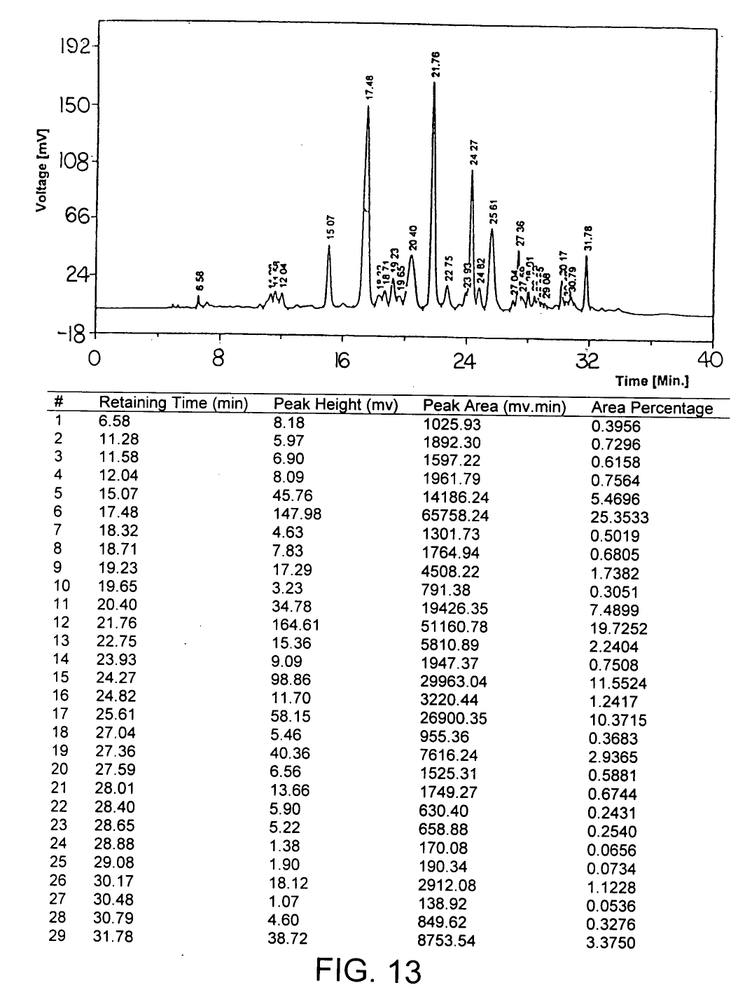

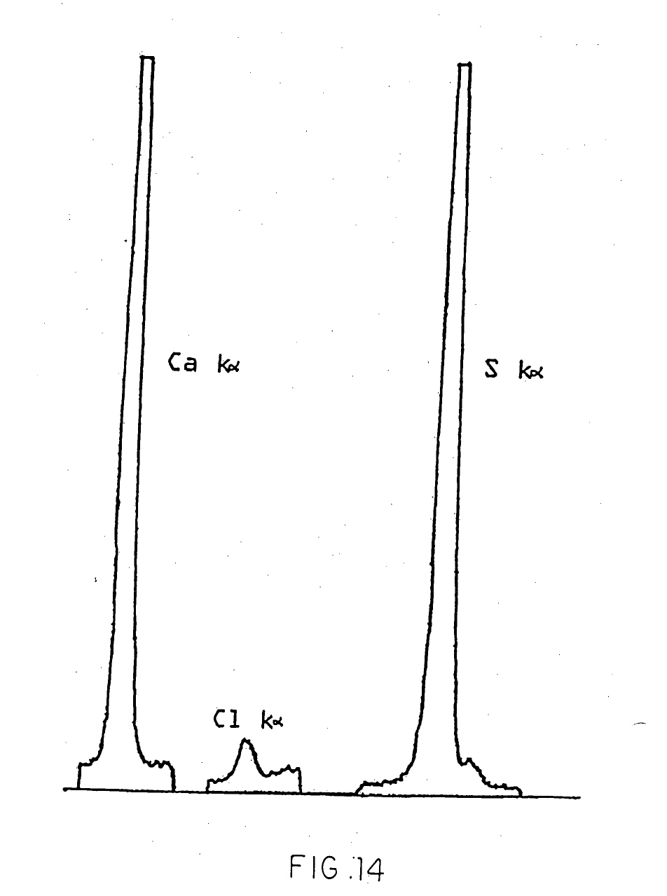

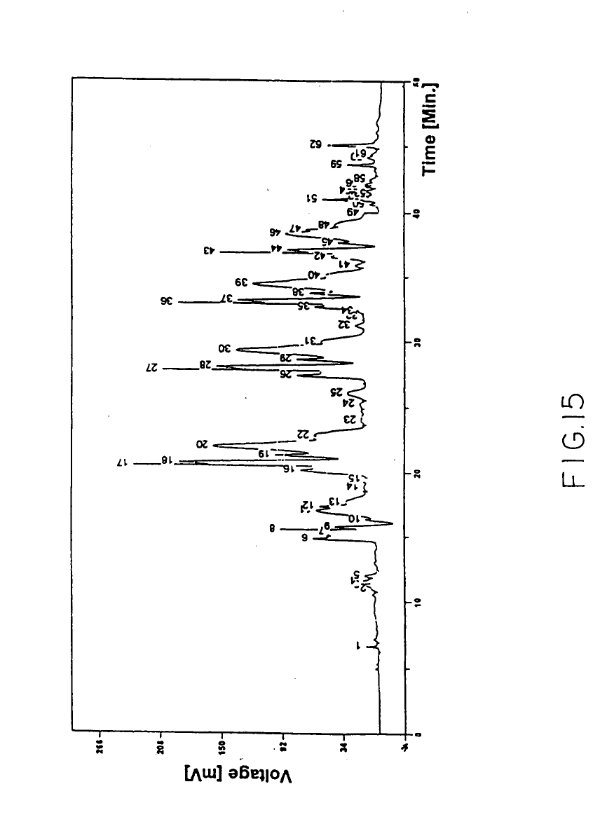

Referring to FIGS. 1 to 15, the present invention provides a manufacturing method and use of broad-spectrum antiviral pharmaceutical composition which is described with two embodiments, extracted from Stellaria media (L.) cyr. or Stellaria L. plants comprising total (organic) phenolic acid glycosides and/or salts of total (organic) phenolic acid glycosides as well as their condensate compounds.

The present invention is to provide a broad-spectrum antiviral pharmaceutical composition which is an extract Chinese herb composition, consisting of total (organic) phenolic acid glycosides, salts of total (organic) phenolic acid glycosides and condensate compounds of the total phenolic acid glycosides extracted from Stellaria media (L.) Cyr. and Stellaria L. plants. This composition containing includes elements of sulfur, nitrogen, and calcium and so on.

The MW 564 component from this composition has been isolated and identified as 6-C-glu, 8-C-ara apigemin glycoside; it is a new compound and called Stellarinol A. In addition, heated with dilute inorganic acid, this composition dilute inorganic acid can turn out a red phlobaphene, so the composition containing flavanoids constituents as well as its condensate.

Hereinafter, composition comprising mixtures of the above extracts is referred to as “Total Stellaria Phenolic Acids Glucosides” (TSPAG). According to the articles of American periodical “Chemical Abstracts” from 1920 to 2002, the chemical constituents of Stellaria L comprise flavone and flavone glycoside, saponin, amidocarbonic acid, chlorogenic, neochlorogenic, ferulic, ethanedioic, caffeic acid, quinic acid, acyl-galactose half lactose lipid, organic alkali, etc. Most constituents mentioned herein are medium polar compound. Those articles did not deal with much about water soluble big polar compound, or introduce intensive research about compound comprising element of sulfur.
The broad-spectrum antiviral pharmaceutical composition is related to big polar compounds extracted from Stellaria L., especially relates to water soluble big polarity compounds comprising nitrogen, calcium and sulfur extracted from Stellaria L.

After intensive patent and article research, no related reports have been found about total phenolic acid glycosides and their condensate compounds as well as its salts of total phenolic acid glycosides from Stellaria L. being used as clinical practice for antiviruses.

The broad-spectrum antiviral pharmaceutical composition is selected from a group of Stellaria L. and Malachium Fries, consisting of Stellaria alsine Grimm, Stellria alsine var. phaeuspetala Hand.-Mazz, Stellaria anhwiensis Migo, Stellaria amblyosepala Schrenk, Stellaria apiculata Wils, 4987 Stellaria aquatica(L) Scop, Stellaria arenaria Maxim, Stellaria arisanensis Hayata, Stellaria arisanensis var. leptophylla Hayata, Stellaria borealis Bigel, Stellaria brahypetala Bge, Stellaria bungeana Fenzl, Stellaria cherleriae(Fisch.) Will, Stellaria chinensis Regel, Stellaria cicrantha, Stellaria crispate Wall, Stellaria davidii Hemsl, Stellaria crispate Wall, Stellaria davidii hemsl, Stellaria decumbens Edgew., Stellaria decunbens var. acicularia Edgew.Et Hook.f., Stellaria delavayi Franch, Stellaria dianthifolia Williams, Stellaria dichasioides Williams, Stellaria dichotoma L., Stellaria dichotoma var. lanceolata Bge., Stellaria dichotoma var. stepheniana Willd., Stellaria diffusa Wills., Stellaria discolor Turcz., Stellaria diversiflora Maxim., Stellaria diversiflora var. gymnandra Franch., Stellaria depressa Schnid., Stellaria duthiei Gandoger, Stellaria filicaulis Mak., Stellaria filipes Komar., Stellaria florida Fisch., Stellaria graminea L., Stellaria graminea var. pilosula Maxim., Stellaria graminea var. viridescens Maxim., Stellaria diversiflora var. gymnandra Franch., Stellaria depressa Schnid., Stellaria duthiei Gandoger, Stellaria filicaulis Mak., Stellaria flipes Komar., Stellaria florida Fisch, Stellaria graminea L., Stellaria graminea var. pilosula Maxim., Stellaria graminea var. viridescens Maxim., Stellaria gyantsensis Williams, Stellaria gypsophiloides Fenzl., Stellaria henryi Williams, Stellaria hsinganensis Kitagawa, Stellaria infracta Maxim., Stellaria leptophylla Hance, Stellaria maximowixziana Franch., Stellaria media (L.) Cyr., Stellaria micrantha Hayata, Stellaria mitans Williams, Stellaria neglecta Weihe, Stellaria neo-palustris Kitagawa, Stellaria octandra Fobedim., Stellaria oxycoccoides Komar., Stellaria palustris L., Stellaria patentifolia Kitagawa, Stellaria pseudosaxatilis Hand.-Mass., Stellaria pusilla Schmid., Stellaria radians L., Stellaria reticulivena Hayata, Stellaria rupestris Hemsl, Stellaria saxatilis Buch-Ham, Stellaria saxatilis var. amplexicaulis Hand.-Mazz., Stellaria soongorica Roshev., Stellaria souliei Williams, Stellaria stellato-pilosa Hayata, Stellaria stongylosepala Hand.-Mazz., Stellaria subumbellata Edgew., Stellaria tomentella Ohwi, Stellaria trimorpha Nakai, Stellaria turkestanica Schischk, Stellaria uda Williams, Stellaria umbellate Truez., Stellaria virdiflora Pax et O. Hoffm., Stellaria wushanensis Williams, Stellaria wutaica Hand.-Mazz., Stellaria yunnanensis franch. J, and Malachium aquaticum (L.) Fries.

According to Chinese herb pharmacology, Stellaria L has the following characteristics:

(i) Nature of drug: sweet with a little bit salty, flat taste, and nonpoisonous.

(ii) Action: activating blood circulation and clearing away blood stasis, mainly for cases of postpartum anemia with dull aching over lower abdomen, insufficient latex, summer heat caused vomiting, periappendicular abscess, gonorrhea, toxic sore, swelling, and beating wound.

(iii) Using direction: for oral use, simmering 50-100 gram, or directly pounding; for external use, pounding and spreading.

According to a first preferred embodiment of the present invention, the manufacturing method of a broad-spectrum antiviral pharmaceutical composition comprises the steps of:

(a) cleaning, powdering and extracting a Setllar.L. plant to form a water extraction;

(b) precipitating, centrifuging and/or filtering the water extraction to obtain a Liquid A;

(c) absorbing mainly TSPAG (total phenolic acid glycosides and their condensate compounds of the Liquid A and/or salt of total phenolic acid glycosides) of the Liquid A on a resin column which is a macroporous resin adsorption column by passing the Liquid A therethrough;

(d) cleaning the resin column by water;

(e) washing the resin column by adding a 5%-60% ethanol to the resin column to obtain and collect a brownish or dark red liquid from the resin column; and

(f) collecting the ethanol from the brownish or dark red liquid and concentrating or vacuum drying the brownish or dark red liquid to form a crude product of the TSPAG, i.e. the total phenolic acid glycosides and/or their condensate compounds and/or salts of total phenolic acid glycosides.

Thereafter, the present invention further comprises a refining process comprising the steps of:

(g) dissolving the crude product of TSPAG by adding water to form a solution;

(h) removing precipitates of the solution by centrifuging;

(i) forming a raw product of TSPAG (raw medicine) by concentrating and drying.

(j) dividing the raw product of TSPAG into three portions: brownish black portion called TSPAG-A, brown portion called TSPAG-B and yellow portion called TSPAG-C by sephadex LH20 column and water.

In view of the nature and property of the raw product of TSPAG of the present invention, the raw product of TSPAG is a kind of hyaloid glassy or porous hyaloid in brown color like most of the dried extract of Chinese traditional medicines. After grinding, it will become a kind of fine brown powder that generally looks like dried extract brown Chinese traditional medicines and smells with Chinese traditional medicines fragrance. It is water soluble, stable condition and has a little bitter taste. Before entering the market on sale, the raw product of TSPAG is preferred to be filtrated through a 0.22 μm membrane filter and/or be radiated by cobalt 56 for disinfection.

According to the first preferred embodiment of the present invention, a practical example is described below to further illustrate the features of the present invention.

In the step (a), a 20 kg Fresh herb of Stellaria L. is fully cleaned, then powdered, centrifuged, filtered, finally extracted to get the water extraction. The residue could be discarded as fertilizer.

In the step (b), the water extraction is precipitated and centrifuged to get the liquid A.

In the step (c), the liquid A is passed through the macroporous resin column (id 10 cm×120 cm) with a flow speed from 1 ml/min to 3 ml/min. It should be observed that from the top to bottom, the color of the resin column gradually changes to brownish red or brownish grey until the whole column has been filled with composition.

In the step (d), the flowed liquid is discarded, and the remaining liquid of resin column is washed by running water thoroughly. Quintuple as much as column volume's distilled water or de-ionized water is added to clean the macroporous resin column. Preferably, approximately 50 liter distilled water is used.

In the step (e), twice as much as column volume's 5% ethanol is first used for cleaning to get some yellow color liquid, discarded. Preferably, approximately 20 liter 5% ethanol is used. Finally, quintuple as much as column volume's 60% ethanol was used to elute and collect the brownish or dark red ethanol solution. Preferably, approximately 50 liter 60% ethanol is used.

In the step (f), the brownish or dark red liquid is concentrated by collecting the ethanol therein by means of a pressure-reduction dryer to form a concentrated viscous liquid, about 100 ml, then the solution is centrifuged (4000 r/min) for 5 minutes to remove the precipitates and the residues are discarded, which is then dried to form the crude product of TSPAG of the present invention, about 49.79 gram.

In the step (g), 200 ml water is added to the crude product of TSPAG to form the solution.

In the step (h), the solution is centrifuged (4000 r/min) for 5 minutes to remove the precipitates and the residues are discarded. The supernatant is filtered trough 0.22 μm filter membrane in a bacteria free container, wherein the solution is concentrated under reduced pressure to form ointment.

In the step (i), the ointment is dried to obtain the fine raw product of TSPAG having a dark brownish red color. After refinement grinding, a total of 48.7 g fine brown powder may be obtained that that generally looks like dried extract brown Chinese traditional medicine and smells with Chinese traditional medicine fragrance. Practically, the yield rate, based on fresh Stellaria, is abort 0.25%. Repeating the above steps may obtain 589.2 g of raw product of TSPAG.

In the step (j), 50 ml water is added to the 100 g of raw product of TSPAG to form the solution, add the solution to top of a sephadex LH20 column(id 5 cm×60 cm) and double as much as column volume's water was used to elute and collect the brownish black color portion called TSPAG-A water solution. Preferably, approximately 2 liter water is used Then, continuingly, double as much as column volume's water was used to elute and collect the brown color portion called TSPAG-B water solution. Preferably, approximately 2 liter water is used. At last, double as much as column volume's 95% ethanol was used to elute and collect the yellow color portion called TSPAG-C ethanol solution. Preferably, approximately 2 liter 95% ethanol is used.

Solutions of TSPAG-A, TSPAG-B and TSPAG-C are concentrated and dried respectively to form the 15.38 gram crude product of TSPAG-A, 66.79 gram crude product of TSPAG-B, and 15.46 gram crude product of TSPAG-C according to the present invention. Practically, the yield rate, based on raw medicine of TSPAG, TSPAG-A is about 15%, TSPAG-B is about 67%, TSPAG-C is about 15%.

The TSPAG-A portion shows the best antiviral effects with more than 2000 Treatment Index by oral or nasal mucosa administration, while the antiviral effect of TSPAG-C portion is much weaker.

According to a second preferred embodiment of the present invention, the manufacturing method of a broad-spectrum antiviral pharmaceutical composition comprises the steps of:

(a) cleaning, powdering and extracting a Setllar.L. plant to form a water extraction;

(b) precipitating, centrifuging and/or filtering the water extraction to obtain a Liquid A;

(c) absorbing mainly total (organic) phenolic acid glycosides of the Liquid A and/or condensate compounds thereof and/or salts of total (organic) phenolic acid glycosides of the Liquid A on an anion exchanging resin column by passing the Liquid A therethrough;

(d) cleaning the anion exchanging resin column by distilled water or de-ionized water;

(e) washing the anion exchanging resin column with sodium chloride solution to obtain and collect effective materials from the anion exchanging resin column; and

(f) desalinating, concentrating and/or filtering the effective materials to form a crude product of total phenolic acid glycoside and/or condensate compounds thereof and/or salts of total phenolic acid glycoside (TSPAG).

Thereafter, the present invention further comprises a refining process comprising the steps of:

(g) dissolving the crude product of TSPAG by adding water to from a solution;

(h) removing precipitates of the solution by centrifuging;

(i) forming a raw product of TSPAG (raw medicine) by concentrating and drying, wherein the raw product of TSPAG.

(j) dividing the raw product of TSPAG into three portions: brownish black portion called TSPAG-A, brown portion called TSPAG-B and yellow portion called TSPAG-C by sephadex LH20 column and water.

According to the second preferred embodiment of the present invention, a practical example is described below to further illustrate the features of the present invention.

In the step (a), a 25 kg Fresh Stellaria L. is fully cleaned, then powdered, centrifuged, filtered, finally extracted to get the water extraction. The residue could be discarded as fertilizer.

In the step (b), the water extraction is precipitated and centrifuged to get the liquid A.

In the step (c), the liquid A is passed through the anion exchanging resin column (id 10 cm×120 cm).

In the step (d), triple as much as column volume's distilled water or de-ionized water is added to clean the ion exchanging column. Preferably, approximately 30 liter distilled water is used. It will be observed that color of flowing liquid from the column gradually change from yellow to tanned, finally flat color.

In the step (e), twice as much as column volume's 2N sodium chloride solution is first used for cleaning to get some dark brownish red color liquid. Preferably, approximately 20 liter saline is used.

In the step (f), the brownish or dark red liquid is concentrated by collecting the sodium chloride solution therein by means of a pressure-reduction and semi-permeable membrane package until the solution is salt-free with running water to an extent being tested inactive with AgNO3 to form a concentrated viscous liquid, about 150 ml, which is then dried to form the crude product of TSPAG of the present invention, around 57.77 gram.

In the step (g), 250 ml water is added to the crude product of TSPAG to form the solution.

In the step (h), the solution is centrifuged (4000 r/min) for 5 minutes to remove the precipitates and the residues are discarded. The supernatant is filtered trough 0.22 μm filter membrane in a bacteria free container, wherein the solution is concentrated under reduced pressure to form ointment.

In the step (i), the ointment is dried to obtain the fine raw product of TSPAG having a dark brownish red color. After refinement grinding, a total of 56.2 g fine brown powder may be obtained that that generally looks like dried extract brown Chinese herb medicine and smells with Chinese herb fragrance. Practically, the yield rate, based on fresh Stellaria, is 2.3%. Repeating the above steps may obtain 311.8 g of raw product of TSPAG.

In the step (j), 50 ml water is added to the 100 g of raw product of TSPAG to form the solution, subsequently, the solution is added to the top of a sephadex LH20 column (id 5 cm×60 cm) and double as much as column volume's water is used to elute and collect the brownish black color portion called TSPAG-A water solution. Preferably, approximate 2 liter water is used. Then double as much as column volume's water is used to elute and collect the brown color portion called TSPAG-B water solution. Preferably, approximate 2 liter water is used. At last, double as much as column volume's 95% ethanol is used to elute and collect the yellow color portion called TSPAG-C ethanol solution. Preferably, approximate 2 liter 95% ethanol is used.

Finally, solutions of TSPAG-A, TSPAG-B and TSPAG-C are then concentrated and dried respectively to form 16.11 gram crude product of TSPAG-A, 67.83 gram crude product of TSPAG-B, and 15.97 gram crude product of TSPAG-C according to the present invention. Practically, the yield rate of, based on raw medicine of TSPAG, TSPAG-A is about 16%, TSPAG-B is about 68%, TSPAG-C is about 16%.

The TSPAG-A portion shows best antiviral effects with more than 2000 Treatment Index by oral or nasal mucosa administration, while the antiviral effect of TSPAG-C portion is the weakest

In view of the nature and property of the crude product of TSPAG of the present invention, the crude product of TSPAG-A is a kind of hyaloid glassy or porous hyaloid in brownish black color, the crude product of TSPAG-B is a kind of hyaloid glassy or porous hyaloid in brown color like most of the dried extract of Chinese traditional medicines, and the crude product of TSPAG-C is a kind of hyaloid glassy or porous hyaloid in yellow color. After grinding, TSPAG-A and B will become a kind of fine brown color powder that generally looks like dried extract brown Chinese traditional medicine and smells with Chinese traditional fragrance. It is water soluble, stable condition and bitter taste. Before entering the market on sale, the crude product of TSPAG A and B is preferred to be filtrated through a 0.22 μm membrane filter and/or be radiated by cobalt 56 for disinfection.

The broad-spectrum antiviral pharmaceutical composition of the present invention, that is the raw product of TSPAG manufactured by either of the above two embodiments comprising total organic phenolic acid/salt of total organic phenolic acid and glycoside thereof, extracted from the Stellaria L, is proven safe and effective and has the following chemical and physical characteristics:

(1) On the basis of colors, TSPAG can be further divided into three portions: brownish black color portion called TSPAG-A, brown color portion called TSPAG-B and yellow color portion called TSPAG-C. The TSPAG-A portion shows best antiviral effects with more than 2000 Treatment Index by oral or nasal mucosa administration. And the antiviral efficacy of TSPAG-C portion is weakest.

(2) TSPAG, especially its brownish black color portion, has an amount of elements of sulfur, nitrogen, and calcium.

(3) TSPAG can be absorbed by DEAE cellulose or anion exchanging agents. TSPAG is acidic.

(4) TSPAG will be changing to dark brown color in case of reacting with ferric trichloride.

The present invention has following advantages effects:

1) TSPAG has shown nontoxic characteristic after in vivo acute toxicity animal test.

a. The maximum tolerance dosage of small mice by nasal mucosa administration (MTD) is 2.25 g/kg.

b. The maximum tolerance dosage of large mice by nasal mucosa administration (MTD) is 1.125 g/kg.

c. The maximum tolerance dosage of small mice by oral administration is 11.25 g/kg.

d. LD50 of TSPAG according to intravenous administration is 3.406 g/kg, its 95% credibility limitation is from 3.069 g/kg to 3.780 g/kg.

2) The present invention didn't show any irritating effects after large mice in vivo experiment by nasal mucosa and oral administration (MTD).

a. There are no toxic effects shown after experiments using different methods such as maximum concentration, maximum administrative capacity, daily once administration, and one week continuous administration. 24 hours after the experiment, the nasal cavities of mice were dissected. There are no symptoms like swelling, ulcers, and congestion has been identified. The mucous membrane condition has shown no difference in comparison with normal saline administration.

b. There are no toxic effects shown after experiments using different methods such as maximum concentration, maximum administrative capacity, daily continuous administration, and one week continuous administration. 24 hours after the experiment, the nasal cavities of mice were dissected. There are no symptoms like swelling, ulcers, and congestion has been identified. The mucous membrane condition has shown no difference in comparison with normal saline administration.
Above information illustrate that TSPAG is one kind of nature medicine with extreme safety, since there are no obvious toxicity and irritation.

3) The present invention has shown antiviral effectiveness: TSPAG having strong broad spectrum antiviral effects.

A. The developed experiments have shown that TSPAG can effectively inhibit various respiratory tract viruses such as influenza viruses, Parainfluenza viruses, rhinovirus, ECHO virus, herpes viruses belonging to RNA group viruses and DNA group adenovirus. Particularly, TSPAG can strongly inhibit influenza virus, parainfluenzaviruses, and adenoviruses, further supporting TSPAG as one kind of broad spectrum antiviral drugs.

B. According to the small mice in vivo experiment for influenza virus test, TSPAG showed strong inhibiting effectiveness towards influenza virus. The small mouse protection ratio (ED50) is 0.51 mg/kg, for the maximum tolerance dosage of small mice by nasal mucosa administration (MTD) is 2250 mg/kg. So TSPAG' treatment index (TI), based on small mice nasal mucosa administration (MTD) is 4412. (TI=MTD/ED50=2250/0.51=4412) The anti-influenza effectiveness of TSPAG has been fully illustrated because 4412 is so high for TI.

Compared with Chinese medicine Shuang Huang Lian Oral Liquor, ED₅₀ being 866 mg/kg, TSPAG's TI is estimated to be 1700 times better.

Compared with Ribavirin, ED₅₀ being 25 mg/kg, LD₅₀ (small mouse 50% lethal ration) being 2000 mg/kg, TI being estimated 80 (TI_{Ribavirin}=LD₅₀/ED₅₀=80), TSPAG's TI is 56 times better.

In short, TSPAG has shown effective inhibiting characteristics as a broad spectrum antiviral drug. Its practical performance is much better than that of Shuang Huang Lian Oral Liquor and Ribavirin.

C. Clinical practice effects:

According to our statistics, the nasal drop dosage of TSPAG, 5 ml solvent with 0.2 g-0.4 g TSPAG, can fully recover common influenza patient within 2 days. Its concentration scopes from 40 mg/ml-80 mg/ml, 120 drops/5 ml, and each nostril is used two drops for administrative practice. The actual administration of TSPAG is around 6 mg/person-12 mg/person each time. This nasal dosage can be used 30 times within two days from 7 am to 21 am. The administration interval is one time per hour. Based on clinical practice in last six years, more than 100 patients have been treated by TSPAG. No matter how long the patient had caught the influenza, he or she would be gradually relieved from influenza symptoms as well as fully recovered within two practical days after being administrated TSPAG. In two days, influenza patient could resume normal health condition. For instance, within an hour after taking TSPAG, patients could be relieved from headache, nasal congestion, nasal discharge, tears, as well as overall paining; within 24 to 48 hours, TSPAG could reduce fever efficiently. Furthermore, the earlier influenza patient took TSPAG, the more effectiveness TSPAG would have shown. In our statistics, one patient was fully recovered from his serious symptoms overnight, (his symptoms including headache, overall paining, high fever 39.5 C, tearing, and nasal discharging), after used TSPAG before going sleep. He could go to school and resume his job the second day. These cases illustrated that TSPAG was of extreme effectiveness beyond ordinary imagination.

According to theoretical calculation from virology institution, Chinese academy of preventive medicine, ED50 of TSPAG equals to 50 mg/kg. Divided by surface coefficient index 10, the quotient will be 0.05 mg/kg, then multiply weight of people, 60 kg, the final result is 3 mg/per time/per person. If this figure were doubled, it would be 6 mg/per time/per person. This ultimate result could be matched by dosage unit in clinical practice, 6 mg/per time/per person to 12 mg/per time/per person. So above information provide sounding evidence and support of TSPAG's clinical application.

During the long period of time clinical practice, there is no toxicity or distinct side effects shown among patients who have been administrated TSPAG for treating influenza. As mentioned above, the maximum tolerance dosage of small mice experiment by nasal mucosa administration (MTD) is 2250 mg/kg, no toxic symptoms have been identified. Actually, the actual practical dosage is only ranged from 6 mg/per time/per person to 12 mg/per time/per person, the total dosage in two days therapeutic period is around 0.2 g to 0.4 g. It is impossible to cause any toxic and side effects.

D. Cellular experiment showed TSPAG has been inhibiting effectiveness towards HIV- 1.

E. Primary stage cellular experiment illustrated TSPAG can inhibit hepatitis virus B and e-antigens.

It is appreciated that the various advantages as well as appreciations of present invention are illustrated by the following Reports and supplemental Tables.

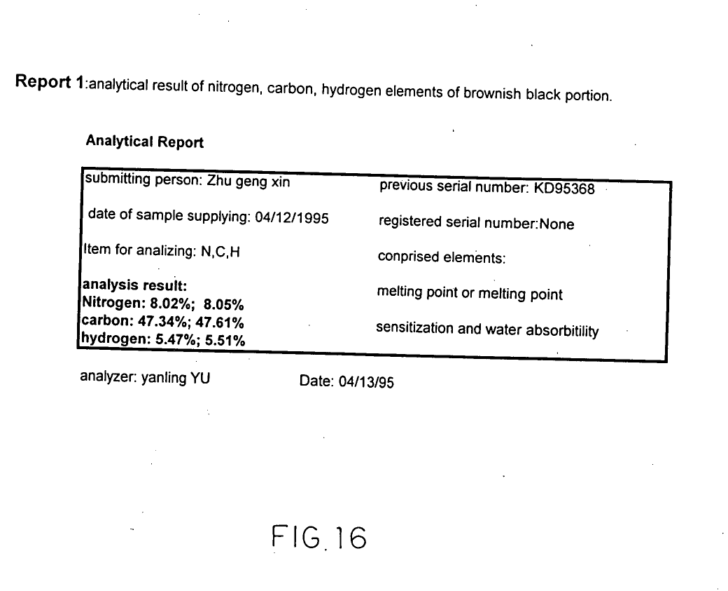

The Report 1 as shown in FIG. 16 shows the analytical result of nitrogen, carbon, hydrogen elements of brownish black portion of TSPAG, which comprise 8% nitrogen, 47% carbon, 5.5% hydrogen.

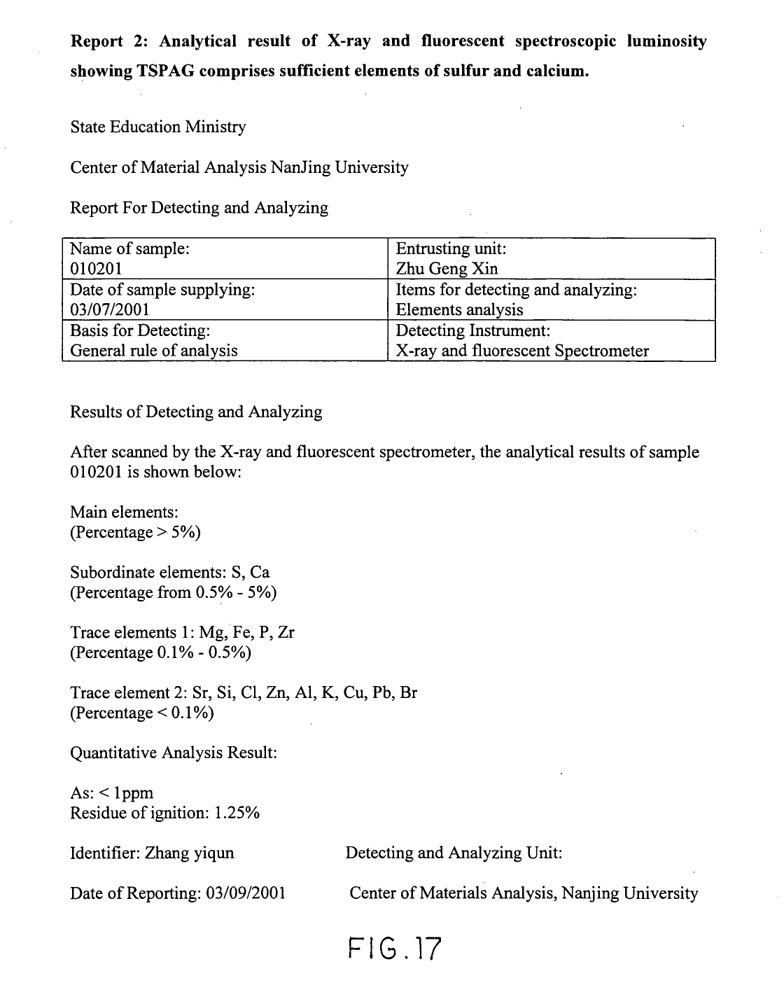

The Report 2 as shown in FIG. 17 shows the analytical result of X-ray and fluorescent spectroscopic luminosity showing TSPAG comprises sufficient elements of sulfur and calcium.

The Report 3 as shown below TSPAG can efficiently inhibit HIV-1 according to cellular experiments.

### Report 3: Cellular Experimental Result for TSPAG's Inhibiting Effects Towards HIV-1

Institute of Virology, Chinese Academy of Preventive Medicine

100 Ying xin yie, xuan wu qu, BeiJing 100052, CHINA Telephone:86-10-63532053 Fax: 86-10-63532053
   Result:
   1, antiviral activity

In vitro experimental result shows that TSPAG is effective for inhibiting HIV, as showing below.

TABLE 1
TSPAG's inhibiting rate towards HIV

Medical concentration:

| | 0.25 | 0.15 | 0.09 | 0.054 |
|---|---|---|---|---|
| Inhibiting | 96.46% | 57% | 3.7% | 0 |
| Rate %: | | | | |

According to statistical calculation, IC₅₀=0.174 mg/ml. Positive contrasting medicines AZT's inhibiting rate is 100% at 1 μg/ml concentration.
2, cellular toxicity

To compare the TSPAG's antiviral activity with cellular toxicity, cellular toxicity has been determined as shown below.

TABLE 1
TSPAG's cellular toxicity

Medical concentration:

| | 5 | 2.5 | 1.25 | 0.625 |
|---|---|---|---|---|
| Cellular lethal | 75 | 42.94 | 17.22 | 0 |
| Rate %: | | | | |

According to statistical calculation, TC₅₀=0.174 mg/ml. Safety index (TC_{50/}/IC₅₀) is 16.7.
Chief Professor: yi Zeng
Professor: zelin Li Date: 08/02/97
Institute of Virology, Chinese Academy of Preventive Medicine

The Report 4 as shown below TSPAG can efficiently inhibit influenza virus, parainfluenza virus, parainfluenza Sendai virus, ECHO virus, and herpes virus belonging to RNA species virus group and adenovirus3, adenovirus7 and adenovirus11 of DNA species virus group.

### Report 4, Experiment Report for TSPAG's Inhibiting Effectiveness in Treating 8 Kinds of Influenzaviruses

Conclusion: TSPAG's inhibiting effects in treating viral illnesses are shown below. The blood clotting result analysis is based on three different methods for treating cellular and chick embryo urinal samples. TSPAG's concentration is 1 mg/ml.

1, TSPAG showed strong inhibiting effectiveness towards adenovirus3 according to three different methods;
2, TSPAG showed strong inhibiting effectiveness in treating rhinovirus according in method 1, HIV according to method 2, 3. In both cases, Infectivity decreased 2 titres.
3, TSPAG could retard the pathological process of adenovirus according method1 and method2; TSPAG could retard the pathological process of ECHO 11 according method 1.
4, TSPAG could inhibit influenza virus effectively by wholly inhibiting virus reproduction according method 1 and reducing the virus reproduction lever 4 times lower.
5, TSPAG could inhibit parainfluenza virus effectively by reducing virus reproduction level 8 times lower according method2.
6, TSPAG showed no effects in treating adneovirus 1.
   In brief, TSPAG showed different inhibiting and killing effects in treating different respiratory viruses according to different treating methods being used. For treating respiratory viruses, except adenovirus 11, method 1 is more effective especially for treating adenovirus 3 and influenza virus 3.

The Report 5 as shown below TSPAG can efficiently inhibit adenovirus3 according to cellular experiment.

### Report 5: Cellular Experimental Result of TSPAG's Inhibiting Effectiveness Towards Adenovirus 3

In vitro experiment of TSPAG's inhibiting effectiveness towards adenovirus.

1, medicine: TSPAG is Chinese herb extract supplied by state hospital.
2, virus: adenovirus 3
3, cell: lung cell of human embryo
4, method and result:

1) TSPAG' sensibility to cellar toxicity. (medical concentration mg/ml)

| Medical | | | | | |
|---|---|---|---|---|---|
| concentration | Concentration (mg/ml) | | | | |
| Time(hr) | 10 mg/ml | 5 mg/ml | 2.5 mg/ml | 1 mg/ml | 0.5 mg/ml |
| 24 | +−/3 | +−/3 | +−/3 | −/3 | −/3 |
| 48 | ++/3 | ++/3 | +/3 | −/3 | −/3 |
| 72 | +++/3 | +++/3 | ++/3 | −/3 | −/3 |
| 96 | +++/3 | +++/3 | +++/3 | +/3 | +−/3 |

1 mg/ml is nontoxic concentration

2) TSPAG's inhibiting effects towards adenovirus 3.

| concentration | | | | | | Virus |
|---|---|---|---|---|---|---|
| virus contrasting | concentration | | | | | affection |
| Time(hr) | 1 mg/ml | 0.5 mg/ml | 0.25 mg/ml | 0.125 mg/ml | 0.0625 mg/ml | contrasting |
| 24 | −−/3 | −/3 | −/3 | −/3 | +−/3 | ++/3 |
| 48 | −/3 | +−/3 | +−/3 | +/3 | +/3 | +++/3 |
| 72 | −/3 | ++/3 | ++/3 | ++/3 | +++/3 | ++++/3 |
| 96 | +−/3 | ++/3 | +++/3 | +++/3 | +++/3 | ++++/3 |

Conclusion: 1 mg/ml concentration TSPAG could effectively inhibit Adenovirus 3.
Microbe research institution of Nanjing medical School.
Jul. 18, 1996

The Report 6 as shown below TSPAG can efficiently inhibit surface antigen of hepatitis B virus and e antigen.

### Report 6: Laboratory Report of TSPAG' Inhibiting Effectiveness in Treating Hepatitis B

Analysis of TSPAG' inhibiting effectiveness towards HBeAg and
HBeAg inside cultured cell 2.2.15

| Experi- | | | | | |
|---|---|---|---|---|---|
| ment | toxicity | HBeAg | | HBeAg | |
| Batch | TC50(mg/ml) | TC50(mg/ml) | SI | TC50(mg/ml) | SI |
| 1 | 2 | 0.69 | 2.90 | 0.713 | 2.805 |

Reporter: Professor hongshan Chen
Experiment summary: li Teng
Experiment Date: February to March, 1999
Location of original experimental data: Insitute of biology, Chinese Academy of Medicine.

The Report 7 as shown below TSPAG can efficiently inhibit vesicular stomatitis virus.

### Report 7: Laboratory Report of TSPAG' Inhibiting Effectiveness in Treating Vesicular Stomatitis Virus

Virus Research Institute Hubei Medical University
Wuhan, Hubei, China
Tel: 86-27-811495
   Method and experimental result:

Different dilutions of TSPAG were added into culture plate with human embryo pneumonocyte. And then TSPAG was discarded after 24 hours, 100TCD₅₀ CVSV (vesicular stomatitis virus) was added to attach pneumonocyte and the contrasting experimental group were applied to test medical effects. 24 hours later, pathologic process of pneumonocyte was observed to obtain the final result shown below.

| TSPAG's | TSPAG's dilution | | | | | | | | | |
|---|---|---|---|---|---|---|---|---|---|---|
| Serial No. | 1/10 | 1/20 | 1/40 | 1/80 | 1/160 | 1/320 | 1/640 | 1/1280 | VSV | normal |
| 0 | + | + | ++ | +++ | +++ | ++++ | ++++ | ++++ | ++++ | 0 |
| 2 | + | + | + | ++ | +++ | +++ | ++++ | ++++ | ++++ | 0 |
| 3 | 0 | 0 | + | + | ++ | ++ | ++++ | ++++ | ++++ | 0 |
| 4 | 0 | 0 | 0 | 0 | + | + | ++ | ++++ | ++++ | 0 |

Clinical Virus Center of Institute of Virology Apr. 28, 1992

The Report 8 as shown below the experimental result of in vivo acute poisonous experiment for animals.

### Report 8: Laboratory Report of TSPAG' Inhibiting Effectiveness Based on In Vivo Acute Poisonous Experiments of Animals

Chinese Herb (I), Documents serial number 15
TSPAG Nasal Drops
Part 3 Pharmacology and toxicity documents
Acute poisonous experiment documents and abstract documents
   Title of Experiment:

Acute poisonous experiment of small mice by gastric administration.

Acute poisonous experiment of small mice by intravenous administration.

Acute poisonous experiment of small mice by nasal administration.

Acute poisonous experiment of large mice by nasal administration.

Topical irritation experiment of large mice by nasal administration.
Experiment supervisor: Associate Professor shuqin Yu
Experiment attendants: pingping Li, zhengfu Xie, meiying Zhang.
Experiment Date: 03/2001 -05/2001
Location of original documents: Pharmaceutical University of China.
Contacted Person: Shuqin Yu
Telephone: 025-3271262
Experiment carrier: Pharmacology research center of Pharmaceutical University, China.
Experimental and abstract documents: Acute poisonous experiment using TSPAG nasal drops.
   1, Abstract

TSPAG has shown nontoxic property after acute poisonous in vivo animal experiment. The maximum tolerance dosage of small mice by gastric administration is 11.25 g/kg. The maximum tolerance dosage of small mice by schneiderian membrane administration (MTD) is 2.25 g/kg. The maximum tolerance dosage of small mice by intravenous administration is 3406 mg/kg. Its 95% credibility limitation is from 3.069 g/kg to 3.779 g/kg.

There are no toxic reaction shown after experiments using different methods such as maximum concentration, maximum administrative capacity, daily once administration, and one week continuous administration. 24 hours after the experiment, the nasal cavities of mice were dissected. There are no symptoms like swelling, ulcers, and congestion have been identified. The mucous membrane condition had shown no difference in comparison with normal saline administration.

2, Objective of experiment

The objective of this experiment is to evaluate the acute poisonous effects and topical irritation of TSPAG nasal drops, based on different administrating methods and different mice.

3, Tested medicine

TSPAG nasal drops were supplied by Nanjing xinrui medical company with batch number 20010303, 450 mg/ml and supposed normal temperature preservation.

4, Animal

Mice of kunming species, were supplied by animal experiment center, Southeast University. The animal quality certificate of Jiang su province is 97002. The ages of small mice are around 40 days, and weights of small mice are around 20+−2 g. And small mice are male/female evenly chosen for this experiment. Each experiment needs 10-20 mice. Accordingly, large mice were male/female evenly chosen too and have 220+−20 g weights.

5, various experiments, dosage units, administration methods

5.1 Experiment of TSPAG' maximizes tolerance dosage of small mice according to gastric administration.

TSPAG was administrated as maximized concentration (450 mg/ml), maximized administrative capacity (0.5 ml/20 g), once daily to observe the toxicity reaction and lethal rate. One dosage batch was used; dosage is 11.25 g/kg.
   5.2 Experiment of TSPAG' maximizes tolerance dosage of small mice according to schneiderian membrane administration.
TSPAG was administrated with maximized concentration (450 mg/ml), maximized administrative capacity (0.1 ml/20 g), once daily to observe the toxicity reaction and lethal rate. One dosage batch was used; dosage is 1.25 g/kg, which is 50 times higher clinical dosage to be used.
   5.3 Experiment: acute poisonous effects of TSPAG' nasal drop based on intravenous administration.
According to randomized methods, a group of testing animal was divided into 5 subgroups. And then administrating dosage was determined according to gradually decreasing method, 1:0.9 subsequently. Each subgroup of small mice was feed with same volume (0.5 ml).
As a result, the administrating dosages of each subgroup are 5000, 4500, 4050, and 3645.
   5.4. Experiment of TSPAG' maximizes tolerance dosage of large mice according to schneiderian membrane administration. TSPAG was administrated with maximized concentration (450 mg/ml), maximized administrative capacity (0.5 ml/20 g), once daily to observe the toxicity reaction and lethal rate.
   5.5 Large mice' irritation experiment of TSPAG by schneiderian membrane administration
TSPAG was administrated with maximized concentration, maximized administrative capacity, once daily. 24 hours later, mice were killed to observe the nasal cavity mucous irritation by naked eyes. Another group of mice were administrated by saline to contrast the experimental result.
There are no toxic symptoms have been identified after mice were continuously administered 24 hours with largest administrative capacity, largest concentration. After the last time administering, the mice were killed to observe the nasal cavity mucous irritation by naked eyes. Another group of mice were administrated by saline to contrast the experimental result.
   6. Pretesting
All experiments were pretested. 15 of mice, divided into 3 groups, have been chosen as pretesting samples for intravenous administration. 3 groups of mice were administered with high dosage, medium dosage and low dosage respectively. The high dosage is 9 g/kg. The lethal rate of mice have been observed to set the administrating dosage standard of formal experiment. For other administrating methods, largest concentration, largest volume dosage have been administrated to 5 mice to observe the lethal rate within one week to confirm the possibility of maximized tolerance testing.
   7. Condition of Experiments

The temperature of laboratory was set to 18-25 degree C. Every 5 of same sex small mice were feed in a 29 cm*18 cm*16 cm cage; large mice were feed in a 29 cm*30 cm*45 cm cage. All mice were feed nutritionally and supplied with fresh water. Padding materials were wood savings and feeding standard is on normal level.

8. Observation Index

Animal' toxicity reaction and lethal rate were observed within 2 weeks. For necessary reasons, mice might be anatomy to observe pathologic process of visceral organs. Accordingly, irritation test could be observed by topical mucous irritating symptoms.

9. Data Process

LD₅₀ was calculated by Bliss method according to the lethal rate in acute poisonous experiment.

10. Result.

10.1 experiment of maximized tolerance of small mice by gastric administration for TSPAG nasal drops.

TSPAG was administrated once in 24 hours with largest concentration (450 mg/ml), largest volume (0.5 ml/20 g). There are no toxic reactions shown among mice, nor mice death occurred after the testing. The maximized tolerance is 11.25 g/kg.

10.2 experiment of maximized tolerance of small mice by schneiderian membrane administration for TSPAG nasal drops. TSPAG was administrated once in 24 hours with largest concentration (450 mg/ml), largest volume (0.1 ml/20 g). There are no toxic reactions shown among mice, nor mice death occurred after the testing. The maximized tolerance is 2.25 g/kg.

10.3 Acute poisonous experiment of small mice by intravenous administration for TSPAG nasal drops. The toxic symptoms of small mice after being injected TSPAG nasal drops include immediate leaping, convulsion, opisthotonus, faster heartbeat, and urinary incontinence. Later, some mice showed symptoms like reduced activities, crouching, feat breath, righting reflex disappearance, and quiver. Particularly in high dosage administration group, the lethal rate is closely related to cardiovascular toxicity. For surviving mice, they eat less and loss weight during the early period of experiment. During the later period, they recovered the weight as well as normal condition. All death occurred in the early four hours exclusively.

The result further illustrated that LD₅₀ of TSPAG nasal drop by intravenous administration is 3406 mg/kg. The 95% confidence limit boundary is 3069-3779 mg/kg.

TABLE 15-1
LD50 of small mice intravenous administration by TSPAG nasal drop

| | | | | | | | | | | Confidence |
|---|---|---|---|---|---|---|---|---|---|---|
| Dosage | Logarithm | Animal | Lethal statistics | | | | Death | Lethal | | limit of |
| (mg/kg) | dosage | quantity | within 2 weeks | | | | quantity | rate % | probablity | LD50 |
| 5000 | 3.699 | 10 | 10 | 0 | 0 | 0 | 10 | 100 | 7.400 | 3406 |
| | | | | | | | | | | 3069-3779 |
| 4500 | 3.653 | 10 | 9 | 0 | 0 | 0 | 9 | 90 | 6.282 | |
| 4050 | 3.608 | 10 | 8 | 0 | 0 | 0 | 8 | 80 | 5.842 | |
| 3645 | 3.562 | 10 | 7 | 0 | 0 | 0 | 7 | 70 | 5.524 | |
| 3280 | 3.516 | 10 | 4 | 0 | 0 | 0 | 4 | 40 | 4.747 | |

10.4 experiment of maximized tolerance of large mice being administrated TSPAG nasal drop.

TSPAG was administrated to large mice once in 24 hours with largest concentration (450 mg/ml), largest volume (0.5 ml/200 g). There are no toxic reaction observed, nor did any mice death occur. The maximized tolerance is 1.125 g/kg.

10.5 experiment of irritation of large mice being administrated TSPAG nasal drop. TSPAG was administrated to large mice once in 24 hours with largest concentration, largest volume. There are no toxic reaction occurred. 24 hours later, mice were killed, and the nasal cavity was anatomy to be observed by naked eyes. There are no congestion and mucous irritation identified.

TSPAG was administrated to large mice continuously within 24 hours with largest concentration, largest volume. There are no toxic reaction occurred. 24 hours later, large mice were killed and the nasal cavity was dissected to be observed by naked eyes. There are no congestion and mucous irritation identified.

11. Conclusion.

Acute poisonous experiments further illustrate that large mice's maximized tolerance of TSPAG gastric administration is 11.25 g/kg, maximized tolerance of TSPAG nasal administration is 2.25 g/kg, and LD₅₀ of TSPAG intravenous administration is 3406 mg/kg. TSPAG' 95% confidence limit boundary is from 3069-3779 mg/kg. The maximized tolerance of small mice by TSPAG gastric administration is 1.125 g/kg.

There are no toxic reactions or death occurred after TSPAG was once or continuously administrated to mice. 24 hours later, mice were killed and nasal cavity was dissected. There is no congestion or mucous ulcers irritation identified by naked eyes. Compared with contrasting group, in which mice were administrated by normal saline, there are no differences.

The Report 9 as shown below laboratory result of in vivo antiviral experiment.

### Report 9 Mice In Vivo Experiment of TSPAG' Antiinfluenzavirus Effect

Conclusion

After in vivo experiment of mice, it is shown that TSPAG has strong inhibiting effects in treating influenza virus.
Experiment designer: shumin Duan, fengju Xue
Experiment supervisor: shumin Duan
Experiment attendant: shumin Duan, xinsheng Zhao, ruifu Wen, and yingjuan Liang
Preliminary adjustment: shumin Duan, yingjuan Liang
Final adjustment: yingjuan Liang
Composer: yingjuan Liang
Location of original documents: Diagnosis section 2, institute of virology, Chinese academy of preventive medicine.
Address: 1oo, yingxin street, xuan wu district, Beijing 100052
Tel: 86-10-63536459

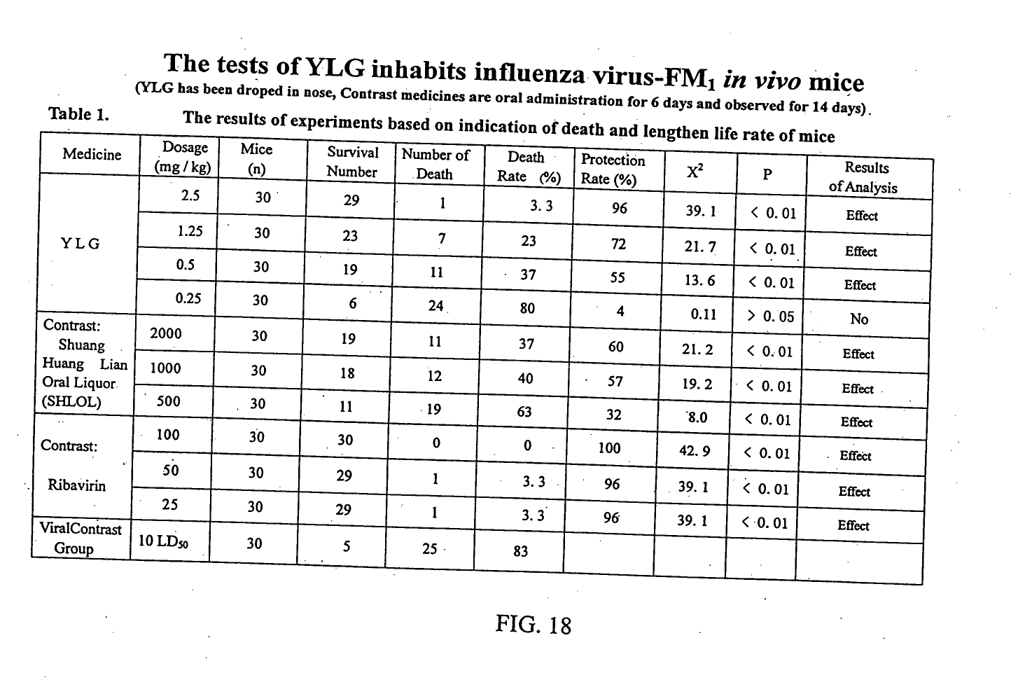

The Table 1 as shown in FIG. 18 illustrates the experimental result of drug effect based on index such as mice lethal rate, protection rate of animal in vivo experiment.

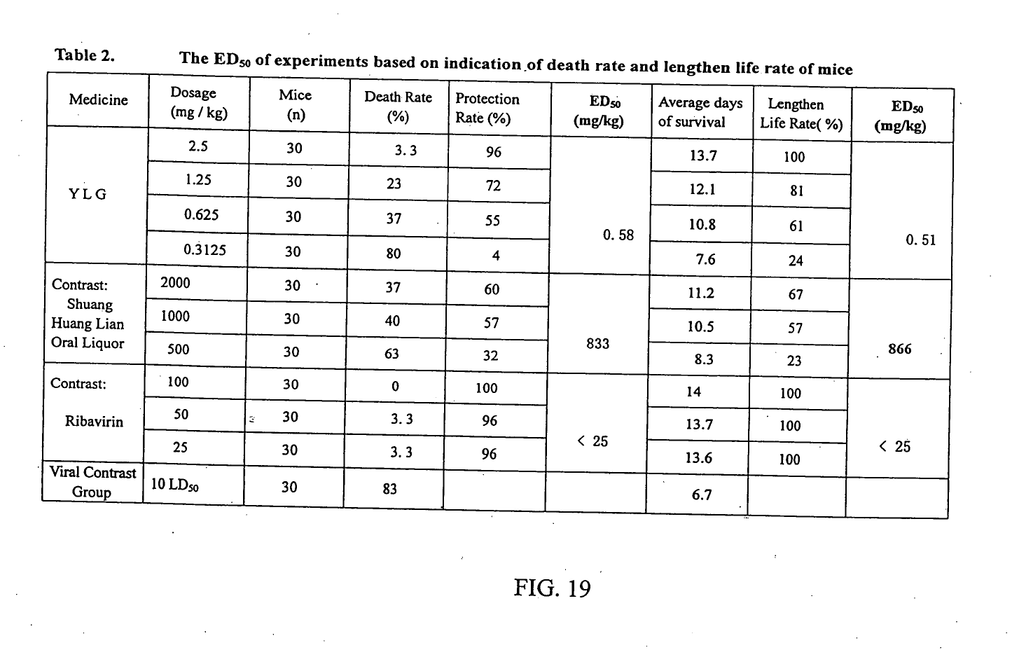

The Table 2 as shown in FIG. 19 illustrates the 50% effective dose (ED₅₀) based on mice protection rate and lives extension rate.

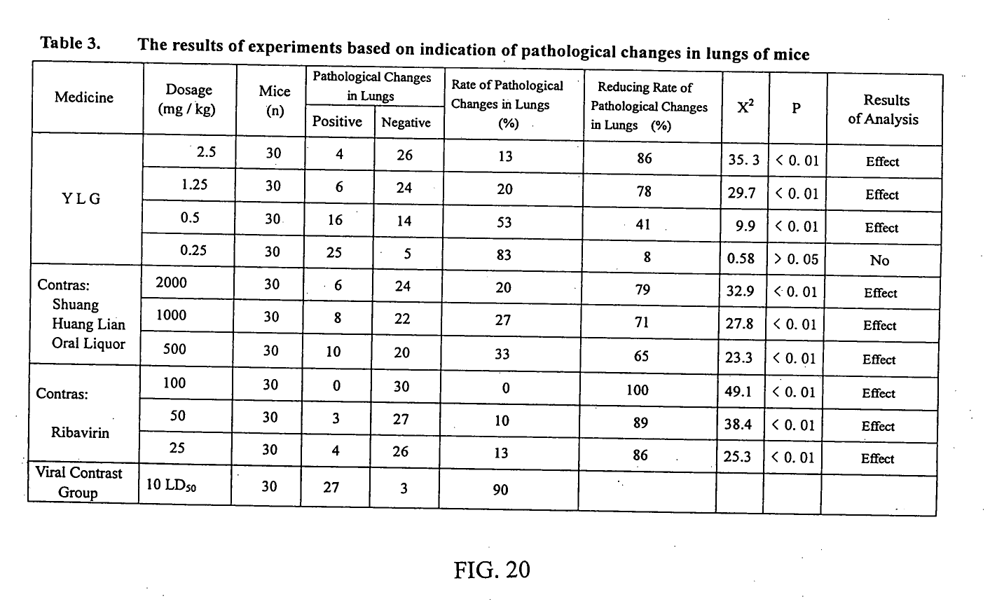

The Table 3 as shown in FIG. 20 illustrates the experimental result of drug effect based on index of pulmo pathologic process of mice

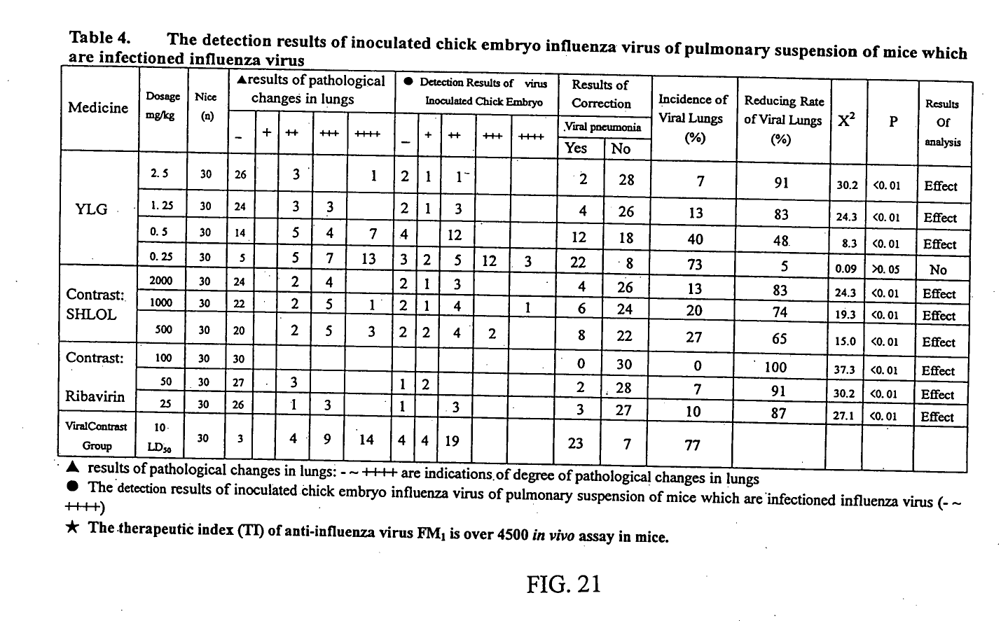

The Table 4 as shown in FIG. 21 is an attached list of Table 3, further illustrating experimental result of drug effect based on index of viral pneumonia, which is affected by mice pneumonocyte suspension being inoculated with fowl embryos.

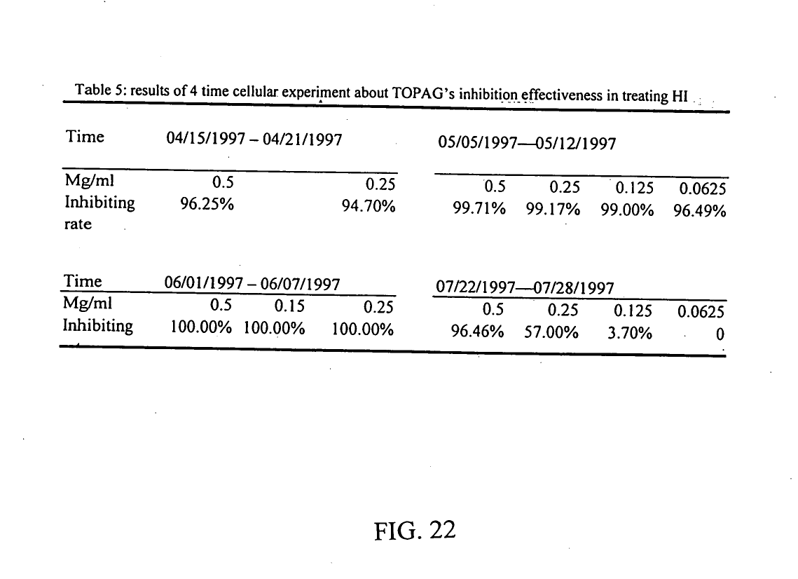

The Table 5 as shown in FIG. 22 illustrates the result of four times cellular experiment about TSPAG's inhibiting effectiveness towards HIV, wherein the fourth experiment is specifically referred to Report 3. It is thus clear that TSPAG, with 60 g/ml concentration, can inhibit almost all HIV.

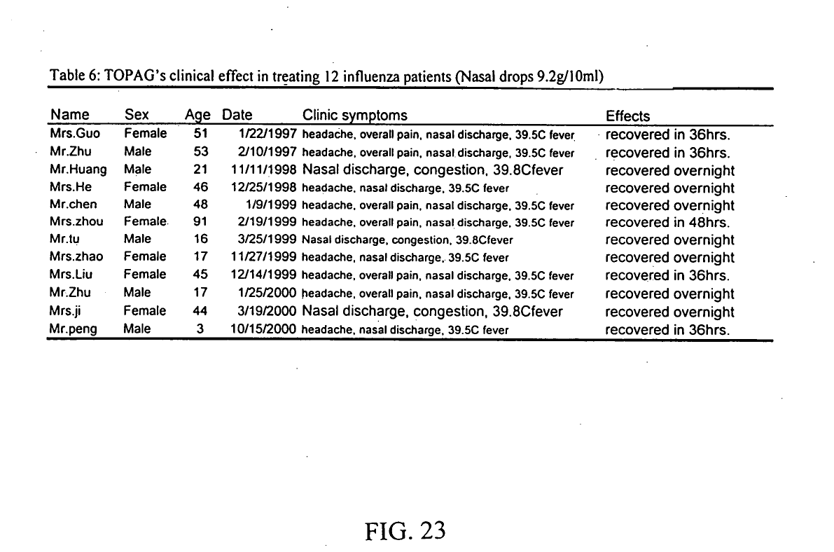

The Table 6 as shown in FIG. 23 illustrates TSPAG's clinical effects: TSPAG at least could reduce 5 days treatment period. Therefore, TSPAG is effectively adapted for treating and preventing influenza. Its therapeutic effect is much better in comparison with neuraminic acid enzyme inhibitor such as Zanamivir and Oseltamivir and qualified for highly efficient drug standard.

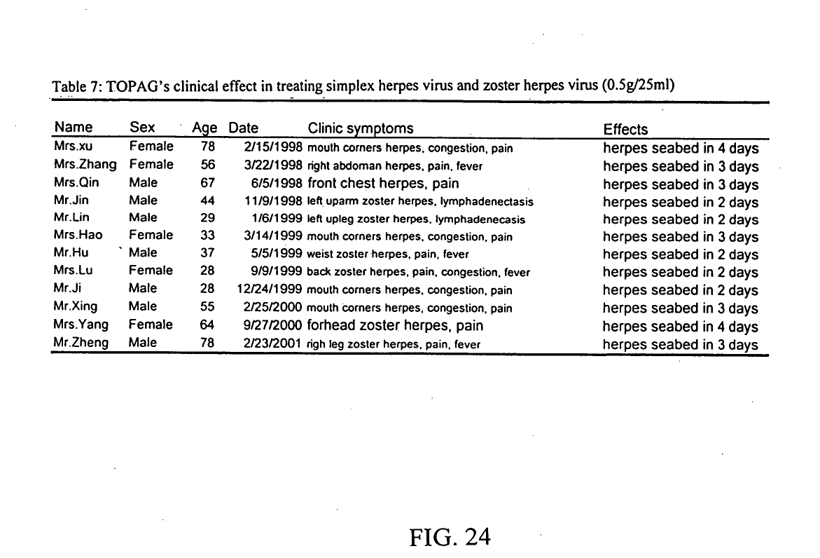

The Table 7 as shown in FIG. 24 illustrates TSPAG's actual effect for clinical practice in treating simplex herpes virus and zoster herpes virus. It is shown that after 0.3 g-0.6 g dosage unit TSPAG were constantly smeared on afflicting area, patients can be relieved from paining, herpes sign will be disappeared, and afflicting area will be seabed within 3 days. It is worth to mention that the concentration of TSPAG is closely related to herpes afflicting size.

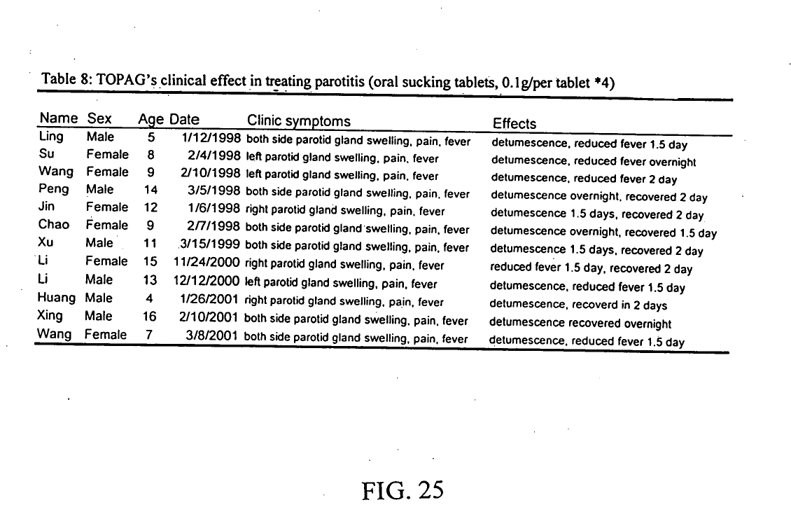

The Table 8 as shown in FIG. 25 illustrates TSPAG's effect in treating parotitis of children. It is shown that in two days, patients can be recovered after being treated by PA sucking tablets comprising total organic phenolic acid as well as its glycosides, with dosage unit ranged from 0.1 g×4 to 0.1 g×6.

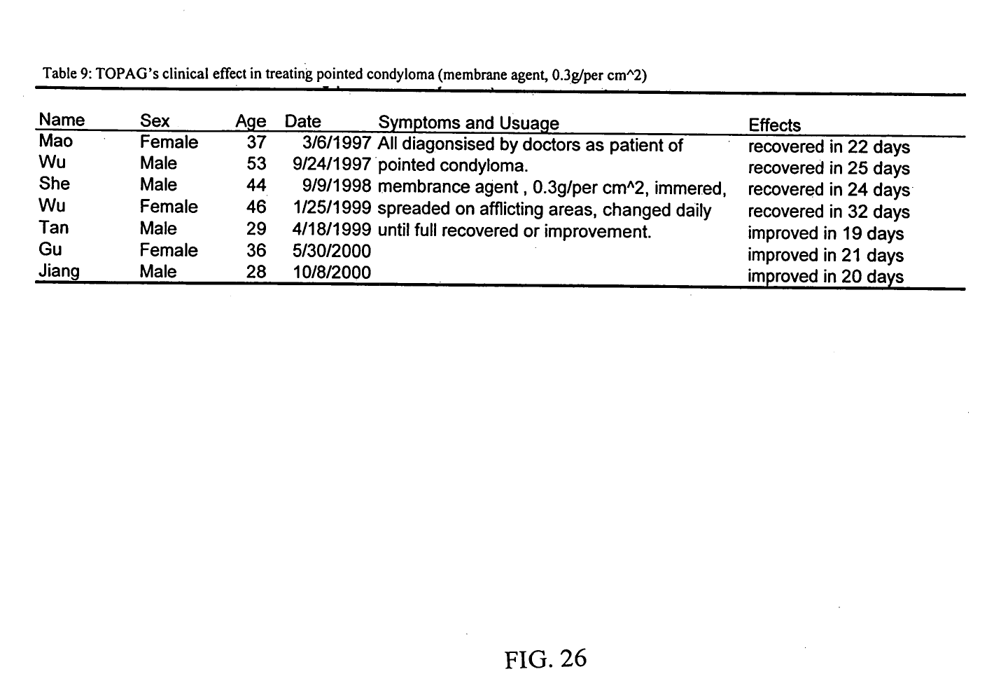

The Table 9 as shown in FIG. 26 illustrates TSPAG' effect in treating venereal disease pointed condyloma. It is rather effective.

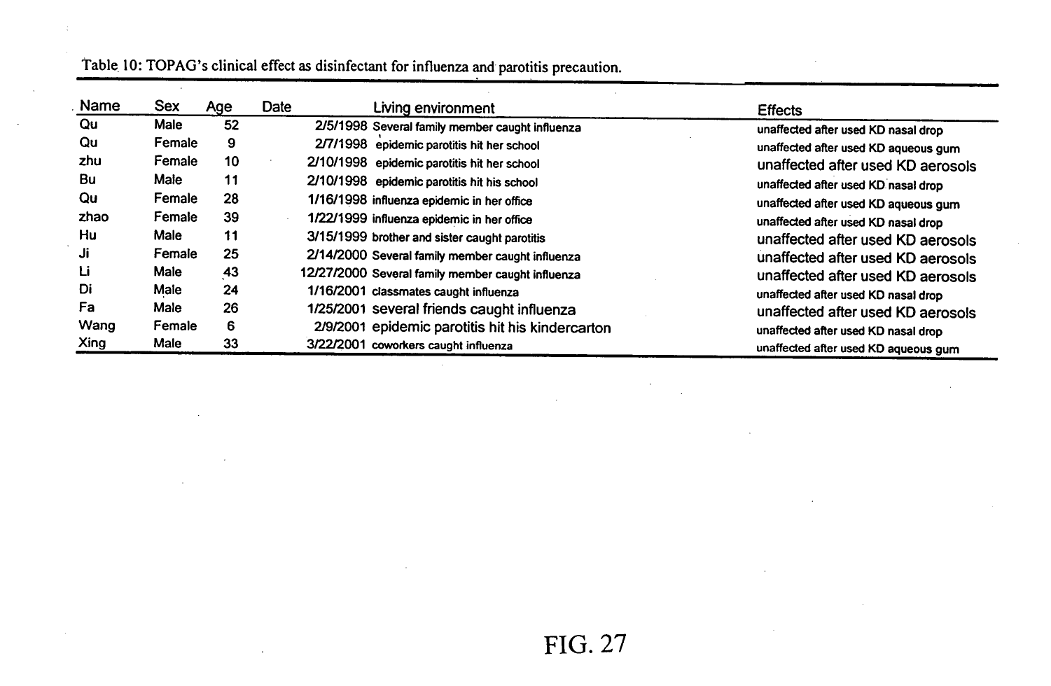

The Table 10 as shown in FIG. 27 illustrates the sterilized effectiveness of the present invention used as disinfectant for influenza and parotitis prevention.

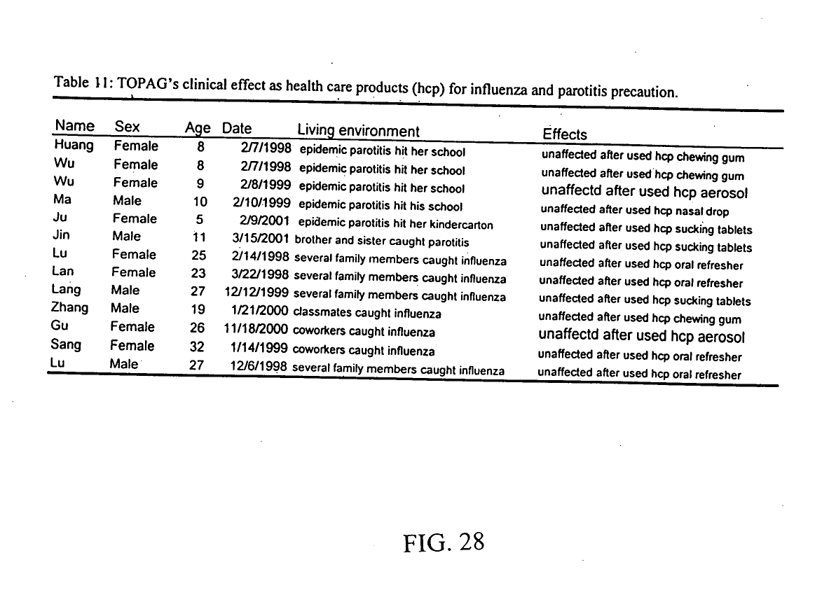

The Table 11 as shown in FIG. 28 illustrates the embodiment of the present invention used as health care products for influenza and parotitis prevention.

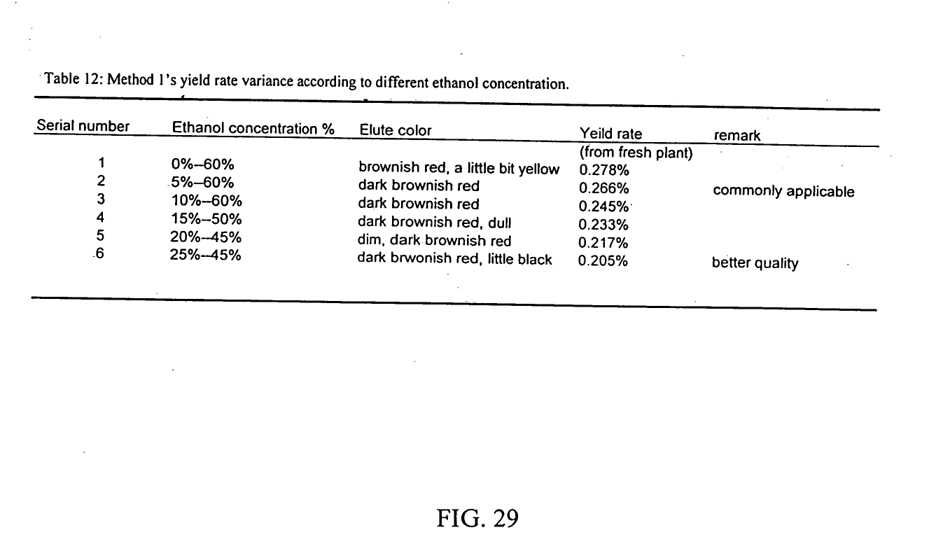

The Table 12 as shown in FIG. 29 illustrates the difference of obtaining rate of various ethanol concentrations used in the above first preferred embodiment.

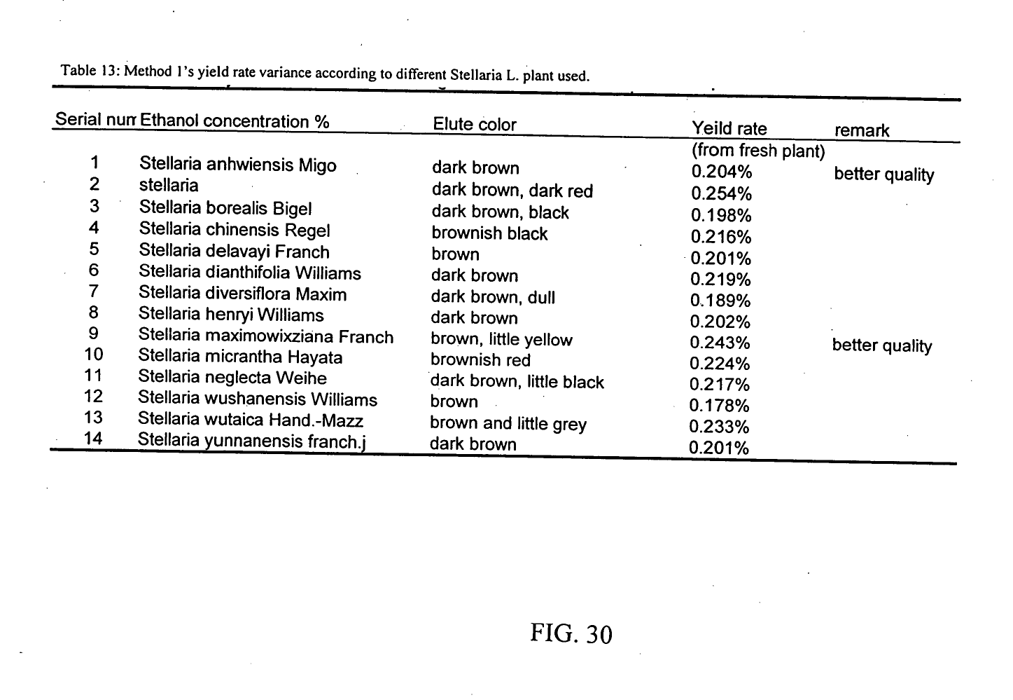

The Table 13 as shown in FIG. 30 illustrates the difference of obtaining rate according to different Stellaria L. used in the above first preferred embodiment.

Various dosage forms of administration, showed below, can be resorted to treat patient using the broad-spectrum antiviral pharmaceutical composition which is the total organic phenolic acid and/or salt of total organic phenolic acid and its glycoside extracted from plant Stellaria L or of Stellaria group.

1. Injection, adapted for treating AIDS, Hepatitis and various viral illnesses, which is produced by the steps of:

(i) dissolving 200 g fine product of the present invention such as obtained from the above first preferred embodiment with 2000 ml 0.9% sodium chloride injection liquid to form an injection solution;

(ii) centrifuging the injection solution for 5 minutes (4000 r/min) to form a centrifuged solution; and

(iii) removing supernatant of the centrifuged solution and sealedly filtering the centrifuged solution with a 0.22 um membrane film in a sterilized uncontaminated container to form the injection product which is filled into injection oriented ampoule, in which the TSPAG could be made into different dosage unit, such as 1 ml/per unit, 0.1 g/per unit, 2 ml/per unit, 0.2 g/per unit respectively after being radiated by element Coluant 56 for sterilization.

2. Powder injection, adapted for treating AIDS, hepatitis, and various viral illnesses, which is produced by the steps of:

(i) dissolving 200 g fine product of the present invention such as obtained from the above first preferred embodiment with 2000 ml distilled water to form a dissolved solution;

(ii) centrifuging the dissolved solution for 5 minutes (4000 r/min) to form a centrifuged solution;

(iii) removing supernatant of the centrifuged solution and sealedly filtering the centrifuged solution with a 0.22 um membrane film in a sterilized uncontaminated container to form an injection solution; and

(iv) drying the injection solution into powder by pulverization so as to be filled into ampoule, wherein the present invention could be made into 2 ml powder dosage unit after being radiation by element Coluent 56 for sterilization. It is worth to mention powder injection should be dissolved with water before being used.

3. Aerosol, adapted for treating influenza and parotitis of new born, disinfectant of air and mouth, as well as health care and antiviral foods, which is produced by the steps of:

(i) filling 4 g fine product of the present invention extracted from the Stellaria L. into 100 ml 0.9% sodium chloride injection liquid to form an injection solution;

(ii) centrifuging the injection solution for 5 minutes (4000 r/min) to form a product solution; and

(iii) filtering the product solution into one or more 10 10 ml volume aerosol bottles respectively. The aerosol could be used as oral disinfectant or health care as well as antiviral foods.

4. Oral or chewing tablets, adapted for treating parotitis, disinfectant of air and mouth, as well as health care and antiviral foods, which is produced by the steps of:

(i) mixing 100 g fine product of the present invention extracted from Stellaria L. such as the composition manufactured in the above first preferred embodiment with dressing, splice, and taste correcting agent to form a mixture; and

(ii) making the mixture into 1000 tablets (1000 mg/per tablet) by tablet machines. It could be used as oral disinfectant, health care or antiviral foods.

5. Capsules, adapted for treating AIDS, hepatitis, influenza and various viruses caused illnesses, which is produced by vacuuming and grinding the fine product of present invention such as obtained from the above first preferred embodiment into 100 g fine powder which is then filled into 1000 capsules under uncontaminated condition, 100 mg/per capsule. It is adapted for treating various viral illnesses as well as oral disinfectant, health care, and antiviral foods.

6. Microcapsules, used as alleviating medicines to treat chronic viral illnesses, which is produced by the steps of:

(i) mixing and suspending the fine product of the present invention such as obtained from the above first preferred embodiment 3.6 g paraffin liquid;

(ii) dissolving 10 g albumin glue with 200 ml distilled water to form a dissolved albumin glue;

(iii) boiling the dissolved albumin glue for at least 30 minutes until oxidizing enzyme is discomposed;

(iv) adding warm liquid paraffin into the boiled albumin glue which is then emulsified for 1 to 2 minutes in a compose bruiser to form an oil-in-water emulsion;

(v) adding 200 ml distilled water with 10 g gelatin glue again and then disposing on 60° C. for bath solution;

(vi) mixing and slowly stirring the albumin glue with 1000 ml beaker under a stabilized temperature 40-50° C. to form a glue liquid;

(vii) adding prepared 5% acetic acid solution into the glue liquid wherein the PH value is adjusted to about 4.1 to generate conglomeration to form capsules;

(viii) filling 750 ml distilled water in the capsules;

(ix) cooling down temperature to 5-10 μC. with water bath to congelate the membrane of gelatin glue capsules, wherein microcapsules are in sphere form;

(x) adding 7 ml 37% aldehyda for solidification and stirring for 15 minutes to finalize the microcapsules;

(xi) adding 20% sodium hydroxide to adjust the PH value to about 8.0 and stirring under reduced temperature for one hour;

(xii) centrifuging and washing to remove aldehyda flavor;

(xiii) adding 3% magnesium stearic acid as thinning agent, mixing evenly, and passing through 16-grid mesh sieve, vacuumed, to get microcapsules of TSPAG. The yield rate is 72.2% and 16.6 g microcapsules have been made, 180 mg/g.

7. Nasal drops, adapted for treating influenza, disinfectant of nasal cavity and health care products, which is produced by the steps of:

(i) dissolving 2 g of the fine product of the present invention extracted from the Stellaria L. according to the above first embodiment with 0.9% sodium chloride injection liquid to form an injection solution;

(ii) centrifuging the injection solution for 5 minutes (4000 r/min) to form a supernatant; and

(iii) filtering the supernatant into an uncontaminated container through 0.22 um membranes and filling into 10 uncontaminated plastic eyedropper bottles to form 200 mg/per unit nasal drops.

8. Coating ointments, adapted for treating skin afflictions such as herpes, pointed condyloma, which is produced by the steps of:

(i) adding 30 g fine product of the present invention extracted from Stellaria L. according to the above first embodiment and 0.3 ml azone into 100 ml water to form a solution;

(ii) centrifuging and filtering into the solution into an uncontaminated container through 0.22 um membranes to obtain a centrifuged and filtered solution;

(iii) adding 19.5 g PVA(05-88) to the centrifuged and filtered solution and then stirring and dissolving the mixture;

(iv) coating the mixture on a plate glass to form 100 mm width, 100 mm length, 0.1 mm thick medicated membrane under sterilized circumstance; and

(v) coating the lucifugal cloth sheet membrane on up and down sides of medicated membrane to form cloth sheet membrane as well as coating ointment. The dosage unit is 0.3 g/square cm and is adapted to being sniped according to clinical application.

9. Mucilage, adapted for treating skin affliction such as herpes, which is produced by the steps of:

(i) dividing and filling 2 g Rhizoma bletillae colloid into ten 15 ml dried glass bottles;

(ii) adding 2 ml glycerin into two bottles respectively as dispersant agent, pendulated;

(iii) dissolving chloroform water with fine product of the present invention to form a product solution;

(iv) stirring, centrifuging and filtering the solution into two bottles through 0.22 um membrane and then shaking violently to form 200 mg/bottle mucilage.

10. Water aqua, adapted for treating skin afflictions such as herpes, pointed condyloma, which is produced by the steps of dissolving 10 g powder of fine product of the present invention extracted from Stellaria L. according to the above first preferred embodiment into 250 ml water to make 25 bottles water aqua, 400 mg/per bottle.

11. Aqueous oral refresher, which is used as oral disinfectant, health care and antiviral foods, is produced by the step of adding splice, taste correcting agent into water aqua to make aqueous chewing gum used as oral disinfectant, and health care antiviral foods.

12. Chewing gum, used as oral disinfectant, health care and antiviral foods, which is produced by the step of mixing the present invention with 100 g gum, 0.1 ml mint oil, 1 g pentaglucose as taste correcting agent into chewing gum used as oral disinfectant, antiviral health care foods.

One skilled in the art will understand that the embodiments of the present invention as shown in the drawings and described above are exemplary only and not intended to be limiting.

It will thus be seen that the objects of the present invention have been fully and effectively accomplished. It embodiments have been shown and described for the purposes of illustrating the functional and structural principles of the present invention and is subject to change without departure from such principles. Therefore, this invention includes all modifications encompassed within the spirit and scope of the following claims.

It is worth to mention that WHO once expected China could make some contribution to resolve influenza and senior cutaneous pruritus. Total phenolic acid glycosides and/or their condensate compounds and/or salts of total phenolic acid glycosides extracted from Plant Stellaria L. have been proven safe and effective to resolved influenza, a difficult problem have puzzled the international medical community over a long time of period. Meanwhile, the TSPAG has been supported by preliminary research for its effect in treating illness caused by HIV, hepatitis etc. Its clinical application could be explored into a wider extent after intensive research. Therefore, the TSPAG, one kind of nature herb extracted according to the present invention, is safe, effective, cheap and convenient broad-spectrum antiviral medicines, which have been long desirable all around the world. The present invention would be significantly influential and valuable on antiviral pharmaceutical development and research for human.

By the way, it is worth to mention that methods for extracting TSPAG is scientific, simple, safe, free of special equipment and highly efficient in keeping raw material's property. Moreover, no environmental issues have been arisen. Plant Stellaria L. is available everywhere all around the world and commonly seen as weed. As a result, TSPAG' raw material, Stellaria L. is widespread, cheaper, easy to culture, easy to reproduce, and finally satisfactory the requirement of high production. In short, the present invention turned waste materials into treasury.

1. A broad-spectrum antiviral pharmaceutical composition comprising total organic and phenolic acid glycosides extracted from one or more Stellaria plants.

2. The broad-spectrum antiviral pharmaceutical composition, as recited in claim 1, further comprising condensate compounds of said total organic and phenolic acid glycosides.

3. The broad-spectrum antiviral pharmaceutical composition, as recited in claim 1, further comprising salts of total organic and phenolic acid glycosides extracted from said Stellaria plants.

4. The broad-spectrum antiviral pharmaceutical composition, as recited in claim 2, further comprising salts of total organic and phenolic acid glycosides extracted from said Stellaria plants.

5. The broad-spectrum antiviral pharmaceutical composition, as recited in claim 1, wherein said Stellaria plants are selected from a plant group consisting of Stellaria alsine Grimm, Stellria alsine var. phaeuspetala Hand.-Mazz, Stellaria anhwiensis Migo, Stellaria amblyosepala Schrenk, Stellaria apiculata Wils, 4987Stellaria aquatica(L) Scop, Stellaria arenaria Maxim, Stellaria arisanensis Hayata, Stellaria arisanensis var. leptophylla Hayata, Stellaria borealis Bigel, Stellaria brahypetala Bge, Stellaria bungeana Fenzl, Stellaria cherleriae(Fisch.) Will, Stellaria chinensis Regel, Stellaria cicrantha, Stellaria crispate Wall, Stellaria davidii Hemsl, Stellaria crispate Wall, Stellaria davidii hemsl, Stellaria decumbens Edgew., Stellaria decunbens var. acicularia Edgew. Et Hook. f., Stellaria delavayi Franch, Stellaria dianthifolia Williams, Stellaria dichasioides Williams, Stellaria dichotoma L., Stellaria dichotoma var. lanceolata Bge., Stellaria dichotoma var. stepheniana Willd., Stellaria diffusa Wills., Stellaria discolor Turcz., Stellaria diversiflora Maxim., Stellaria diversiflora var. gymnandra Franch., Stellaria depressa Schnid., Stellaria duthiei Gandoger, Stellaria filicaulis Mak., Stellaria filipes Komar., Stellaria florida Fisch., Stellaria graminea L., Stellaria graminea var. pilosula Maxim., Stellaria graminea var. viridescens Maxim., Stellaria diversiflora var. gymnandra Franch., Stellaria depressa Schnid., Stellaria duthiei Gandoger, Stellaria filicaulis Mak., Stellaria flipes Komar., Stellaria florida Fisch, Stellaria graminea L., Stellaria graminea var. pilosula Maxim., Stellaria graminea var. viridescens Maxim., Stellaria gyantsensis Williams, Stellaria gypsophiloides Fenzl., Stellaria henryi Williams, Stellaria hsinganensis Kitagawa, Stellaria infracta Maxim., Stellaria leptophylla Hance, Stellaria maximowixziana Franch., Stellaria media(L.) Cyr., Stellaria micrantha Hayata, Stellaria mitans Williams, Stellaria neglecta Weihe, Stellaria neo-palustris Kitagawa, Stellaria octandra Fobedim., Stellaria oxycoccoides Komar., Stellaria palustris L., Stellaria patentifolia Kitagawa, Stellaria pseudosaxatilis Hand.-Mass., Stellaria pusilla Schmid., Stellaria radians L., Stellaria reticulivena Hayata, Stellaria rupestris Hemsl, Stellaria saxatilis Buch-Ham, Stellaria saxatilis var. amplexicaulis Hand.-Mazz., Stellaria soongorica Roshev., Stellaria souliei Williams, Stellaria stellato-pilosa Hayata, Stellaria stongylosepala Hand.-Mazz., Stellaria subumbellata Edgew., Stellaria tomentella Ohwi, Stellaria trimorpha Nakai, Stellaria turkestanica Schischk, Stellaria uda Williams, Stellaria umbellate Truez., Stellaria virdiflora Pax et O. Hoffm., Stellaria wushanensis Williams, Stellaria wutaica Hand.-Mazz., Stellaria yunnanensis franch. J, and Malachium aquaticum (L.) Fries.

6. The broad-spectrum antiviral pharmaceutical composition, as recited in claim 2, wherein said Stellaria plants are selected from a plant group consisting of Stellaria alsine Grimm, Stellria alsine var. phaeuspetala Hand.-Mazz, Stellaria anhwiensis Migo, Stellaria amblyosepala Schrenk, Stellaria apiculata Wils, 4987Stellaria aquatica(L) Scop, Stellaria arenaria Maxim, Stellaria arisanensis Hayata, Stellaria arisanensis var. leptophylla Hayata, Stellaria borealis Bigel, Stellaria brahypetala Bge, Stellaria bungeana Fenzl, Stellaria cherleriae(Fisch.) Will, Stellaria chinensis Regel, Stellaria cicrantha, Stellaria crispate Wall, Stellaria davidii Hemsl, Stellaria crispate Wall, Stellaria davidii hemsl, Stellaria decumbens Edgew., Stellaria decunbens var. acicularia Edgew. Et Hook. f., Stellaria delavayi Franch, Stellaria dianthifolia Williams, Stellaria dichasioides Williams, Stellaria dichotoma L., Stellaria dichotoma var. lanceolata Bge., Stellaria dichotoma var. stepheniana Willd., Stellaria diffusa Wills., Stellaria discolor Turcz., Stellaria diversiflora Maxim., Stellaria diversiflora var. gymnandra Franch., Stellaria depressa Schnid., Stellaria duthiei Gandoger, Stellaria filicaulis Mak., Stellaria filipes Komar., Stellaria florida Fisch., Stellaria graminea L., Stellaria graminea var. pilosula Maxim., Stellaria graminea var. viridescens Maxim., Stellaria diversiflora var. gymnandra Franch., Stellaria depressa Schnid., Stellaria duthiei Gandoger, Stellaria filicaulis Mak., Stellaria flipes Komar., Stellaria florida Fisch, Stellaria graminea L., Stellaria graminea var. pilosula Maxim., Stellaria graminea var. viridescens Maxim., Stellaria gyantsensis Williams, Stellaria gypsophiloides Fenzl., Stellaria henryi Williams, Stellaria hsinganensis Kitagawa, Stellaria infracta Maxim., Stellaria leptophylla Hance, Stellaria maximowixziana Franch., Stellaria media(L.) Cyr., Stellaria micrantha Hayata, Stellaria mitans Williams, Stellaria neglecta Weihe, Stellaria neo-palustris Kitagawa, Stellaria octandra Fobedim., Stellaria oxycoccoides Komar., Stellaria palustris L., Stellaria patentifolia Kitagawa, Stellaria pseudosaxatilis Hand.-Mass., Stellaria pusilla Schmid., Stellaria radians L., Stellaria reticulivena Hayata, Stellaria rupestris Hemsl, Stellaria saxatilis Buch-Ham, Stellaria saxatilis var. amplexicaulis Hand.-Mazz., Stellaria soongorica Roshev., Stellaria souliei Williams, Stellaria stellato-pilosa Hayata, Stellaria stongylosepala Hand.-Mazz., Stellaria subumbellata Edgew., Stellaria tomentella Ohwi, Stellaria trimorpha Nakai, Stellaria turkestanica Schischk, Stellaria uda Williams, Stellaria umbellate Truez., Stellaria virdiflora Pax et O. Hoffm., Stellaria wushanensis Williams, Stellaria wutaica Hand.-Mazz., Stellaria yunnanensis franch. J, and Malachium aquaticum (L.) Fries.

7. The broad-spectrum antiviral pharmaceutical composition, as recited in claim 4, wherein said Stellaria plants are selected from a plant group consisting of Stellaria alsine Grimm, Stellria alsine var. phaeuspetala Hand.-Mazz, Stellaria anhwiensis Migo, Stellaria amblyosepala Schrenk, Stellaria apiculata Wils, 4987Stellaria aquatica(L) Scop, Stellaria arenaria Maxim, Stellaria arisanensis Hayata, Stellaria arisanensis var. leptophylla Hayata, Stellaria borealis Bigel, Stellaria brahypetala Bge, Stellaria bungeana Fenzl, Stellaria cherleriae(Fisch.) Will, Stellaria chinensis Regel, Stellaria cicrantha, Stellaria crispate Wall, Stellaria davidii Hemsl, Stellaria crispate Wall, Stellaria davidii hemsl, Stellaria decumbens Edgew., Stellaria decunbens var. acicularia Edgew. Et Hook. f., Stellaria delavayi Franch, Stellaria dianthifolia Williams, Stellaria dichasioides Williams, Stellaria dichotoma L., Stellaria dichotoma var. lanceolata Bge., Stellaria dichotoma var. stepheniana Willd., Stellaria diffusa Wills., Stellaria discolor Turcz., Stellaria diversiflora Maxim., Stellaria diversiflora var. gymnandra Franch., Stellaria depressa Schnid., Stellaria duthiei Gandoger, Stellaria filicaulis Mak., Stellaria filipes Komar., Stellaria florida Fisch., Stellaria graminea L., Stellaria graminea var. pilosula Maxim., Stellaria graminea var. viridescens Maxim., Stellaria diversiflora var. gymnandra Franch., Stellaria depressa Schnid., Stellaria duthiei Gandoger, Stellaria filicaulis Mak., Stellaria flipes Komar., Stellaria florida Fisch, Stellaria graminea L., Stellaria graminea var. pilosula Maxim., Stellaria graminea var. viridescens Maxim., Stellaria gyantsensis Williams, Stellaria gypsophiloides Fenzl., Stellaria henryi Williams, Stellaria hsinganensis Kitagawa, Stellaria infracta Maxim., Stellaria leptophylla Hance, Stellaria maximowixziana Franch., Stellaria media(L.) Cyr., Stellaria micrantha Hayata, Stellaria mitans Williams, Stellaria neglecta Weihe, Stellaria neo-palustris Kitagawa, Stellaria octandra Fobedim., Stellaria oxycoccoides Komar., Stellaria palustris L., Stellaria patentifolia Kitagawa, Stellaria pseudosaxatilis Hand.-Mass., Stellaria pusilla Schmid., Stellaria radians L., Stellaria reticulivena Hayata, Stellaria rupestris Hemsl, Stellaria saxatilis Buch-Ham, Stellaria saxatilis var. amplexicaulis Hand.-Mazz., Stellaria soongorica Roshev., Stellaria souliei Williams, Stellaria stellato-pilosa Hayata, Stellaria stongylosepala Hand.-Mazz., Stellaria subumbellata Edgew., Stellaria tomentella Ohwi, Stellaria trimorpha Nakai, Stellaria turkestanica Schischk, Stellaria uda Williams, Stellaria umbellate Truez., Stellaria virdiflora Pax et O. Hoffm., Stellaria wushanensis Williams, Stellaria wutaica Hand.-Mazz., Stellaria yunnanensis franch. J, and Malachium aquaticum (L.) Fries.

8. The broad-spectrum antiviral pharmaceutical composition, as recited in claim 7, further comprising an element selected from a group consisting of sulfur, nitrogen and calcium.

9. A method of treating a viral illness, including HIV, influenza, hepatitis, parotitis, and pointed condyloma, comprising a step of administrating a broad-spectrum antiviral composition comprising total organic and phenolic acid glycosides extracted from one or more Stellaria plants.

10. The method, as recited in claim 9, wherein said broad-spectrum antiviral composition further comprises condensate compounds of said total organic and phenolic acid glycosides.

11. The method, as recited in claim 10, wherein said broad-spectrum antiviral composition further comprises salts of total organic and phenolic acid glycosides extracted from said Stellaria plants.

12. The method, as recited in claim 9, wherein said Stellaria plants are selected from a group consisting of Stellaria alsine Grimm, Stellria alsine var. phaeuspetala Hand.-Mazz, Stellaria anhwiensis Migo, Stellaria amblyosepala Schrenk, Stellaria apiculata Wils, 4987Stellaria aquatica(L) Scop, Stellaria arenaria Maxim, Stellaria arisanensis Hayata, Stellaria arisanensis var. leptophylla Hayata, Stellaria borealis Bigel, Stellaria brahypetala Bge, Stellaria bungeana Fenzl, Stellaria cherleriae(Fisch.) Will, Stellaria chinensis Regel, Stellaria cicrantha, Stellaria crispate Wall, Stellaria davidii Hemsl, Stellaria crispate Wall, Stellaria davidii hemsl, Stellaria decumbens Edgew., Stellaria decunbens var. acicularia Edgew. Et Hook. f., Stellaria delavayi Franch, Stellaria dianthifolia Williams, Stellaria dichasioides Williams, Stellaria dichotoma L., Stellaria dichotoma var. lanceolata Bge., Stellaria dichotoma var. stepheniana Willd., Stellaria diffusa Wills., Stellaria discolor Turcz., Stellaria diversiflora Maxim., Stellaria diversiflora var. gymnandra Franch., Stellaria depressa Schnid., Stellaria duthiei Gandoger, Stellaria filicaulis Mak., Stellaria filipes Komar., Stellaria florida Fisch., Stellaria graminea L., Stellaria graminea var. pilosula Maxim., Stellaria graminea var. viridescens Maxim., Stellaria diversiflora var. gymnandra Franch., Stellaria depressa Schnid., Stellaria duthiei Gandoger, Stellaria filicaulis Mak., Stellaria flipes Komar., Stellaria florida Fisch, Stellaria graminea L., Stellaria graminea var. pilosula Maxim., Stellaria graminea var. viridescens Maxim., Stellaria gyantsensis Williams, Stellaria gypsophiloides Fenzl., Stellaria henryi Williams, Stellaria hsinganensis Kitagawa, Stellaria infracta Maxim., Stellaria leptophylla Hance, Stellaria maximowixziana Franch., Stellaria media(L.) Cyr., Stellaria micrantha Hayata, Stellaria mitans Williams, Stellaria neglecta Weihe, Stellaria neo-palustris Kitagawa, Stellaria octandra Fobedim., Stellaria oxycoccoides Komar., Stellaria palustris L., Stellaria patentifolia Kitagawa, Stellaria pseudosaxatilis Hand.-Mass., Stellaria pusilla Schmid., Stellaria radians L., Stellaria reticulivena Hayata, Stellaria rupestris Hemsl, Stellaria saxatilis Buch-Ham, Stellaria saxatilis var. amplexicaulis Hand.-Mazz., Stellaria soongorica Roshev., Stellaria souliei Williams, Stellaria stellato-pilosa Hayata, Stellaria stongylosepala Hand.-Mazz., Stellaria subumbellata Edgew., Stellaria tomentella Ohwi, Stellaria trimorpha Nakai, Stellaria turkestanica Schischk, Stellaria uda Williams, Stellaria umbellate Truez., Stellaria virdiflora Pax et O. Hoffm., Stellaria wushanensis Williams, Stellaria wutaica Hand.-Mazz., Stellaria yunnanensis franch. J, and Malachium aquaticum (L.) Fries.

13. The method, as recited in claim 1 1, wherein said Stellaria plants are selected from a group consisting of Stellaria alsine Grimm, Stellria alsine var. phaeuspetala Hand.-Mazz, Stellaria anhwiensis Migo, Stellaria amblyosepala Schrenk, Stellaria apiculata Wils, 4987Stellaria aquatica(L) Scop, Stellaria arenaria Maxim, Stellaria arisanensis Hayata, Stellaria arisanensis var. leptophylla Hayata, Stellaria borealis Bigel, Stellaria brahypetala Bge, Stellaria bungeana Fenzl, Stellaria cherleriae(Fisch.) Will, Stellaria chinensis Regel, Stellaria cicrantha, Stellaria crispate Wall, Stellaria davidii Hemsl, Stellaria crispate Wall, Stellaria davidii hemsl, Stellaria decumbens Edgew., Stellaria decunbens var. acicularia Edgew. Et Hook. f., Stellaria delavayi Franch, Stellaria dianthifolia Williams, Stellaria dichasioides Williams, Stellaria dichotoma L., Stellaria dichotoma var. lanceolata Bge., Stellaria dichotoma var. stepheniana Willd., Stellaria diffusa Wills., Stellaria discolor Turcz., Stellaria diversiflora Maxim., Stellaria diversiflora var. gymnandra Franch., Stellaria depressa Schnid., Stellaria duthiei Gandoger, Stellaria filicaulis Mak., Stellaria filipes Komar., Stellaria florida Fisch., Stellaria graminea L., Stellaria graminea var. pilosula Maxim., Stellaria graminea var. viridescens Maxim., Stellaria diversiflora var. gymnandra Franch., Stellaria depressa Schnid., Stellaria duthiei Gandoger, Stellaria filicaulis Mak., Stellaria flipes Komar., Stellaria florida Fisch, Stellaria graminea L., Stellaria graminea var. pilosula Maxim., Stellaria graminea var. viridescens Maxim., Stellaria gyantsensis Williams, Stellaria gypsophiloides Fenzl., Stellaria henryi Williams, Stellaria hsinganensis Kitagawa, Stellaria infracta Maxim., Stellaria leptophylla Hance, Stellaria maximowixziana Franch., Stellaria media(L.) Cyr., Stellaria micrantha Hayata, Stellaria mitans Williams, Stellaria neglecta Weihe, Stellaria neo-palustris Kitagawa, Stellaria octandra Fobedim., Stellaria oxycoccoides Komar., Stellaria palustris L., Stellaria patentifolia Kitagawa, Stellaria pseudosaxatilis Hand.-Mass., Stellaria pusilla Schmid., Stellaria radians L., Stellaria reticulivena Hayata, Stellaria rupestris Hemsl, Stellaria saxatilis Buch-Ham, Stellaria saxatilis var. amplexicaulis Hand.-Mazz., Stellaria soongorica Roshev., Stellaria souliei Williams, Stellaria stellato-pilosa Hayata, Stellaria stongylosepala Hand.-Mazz., Stellaria subumbellata Edgew., Stellaria tomentella Ohwi, Stellaria trimorpha Nakai, Stellaria turkestanica Schischk, Stellaria uda Williams, Stellaria umbellate Truez., Stellaria virdiflora Pax et O. Hoffm., Stellaria wushanensis Williams, Stellaria wutaica Hand.-Mazz., Stellaria yunnanensis franch. J, and Malachium aquaticum (L.) Fries.

14. A method of producing a broad-spectrum antiviral composition, comprising the steps of:
(a) cleaning, powdering and extracting one or more Stellaria plants to form a water extraction;
(b) producing a liquid from said water extraction;
(c) absorbing total organic and phenolic acid glycosides of said liquid on a resin column by passing said liquid through said resin column;
(d) cleaning said resin column by water;
(e) washing said resin column by adding an agent to said resin column to obtain and collect a brownish or dark red liquid from said resin column; and
(f) collecting said ethanol from said brownish or dark red liquid and forming a crude product containing said total organic phenolic acid glycosides.

15. The method, as recited in claim 14, wherein said agent includes an ethanol.

16. The method, as recited in claim 15, wherein the step (b) comprises a step of precipitating and centrifuging said water extraction to obtain said liquid.

17. The method, as recited in claim 15, wherein the step (b) comprises a step of filtering said water extraction to obtain said liquid.

18. The method, as recited in claim 15, wherein the step (c) further comprises a step of absorbing salts of total organic and phenolic acid glycosides of said liquid, wherein in the step (f), said crude product further contains said salts of total organic and phenolic acid glycosides and said condensate compounds of said total organic and phenolic acid glycosides.

19. The method, as recited in claim 16, wherein the step (c) further comprises a step of absorbing salts of total organic and phenolic acid glycosides of said liquid, wherein in the step (f), said crude product further contains said salts of total organic and phenolic acid glycosides and said condensate compounds of said total organic and phenolic acid glycosides.

20. The method, as recited in claim 17, wherein the step (c) further comprises a step of absorbing salts of total organic and phenolic acid glycosides and condensate compounds of said total organic and phenolic acid glycosides of said liquid, wherein in the step (f), said crude product further contains said salts of total organic and phenolic acid glycosides and said condensate compounds of said total organic and phenolic acid glycosides.

21. The method, as recited in claim 15, wherein said resin column is a macroporous resin column.

22. The method, as recited in claim 20, wherein said resin column is a macroporous resin column.

23. The method, as recited in claim 15, wherein in the step (e), said ethanol is a 5%-60% ethanol.

24. The method, as recited in claim 22, wherein in the step (e), said ethanol is a 5%-60% ethanol.

25. The method, as recited in claim 15, wherein the step (f) comprises a step of concentrating said brownish or dark red liquid to form said crude product.

26. The method, as recited in claim 22, wherein the step (f) comprises a step of concentrating said brownish or dark red liquid to form said crude product.

27. The method, as recited in claim 24, wherein the step (f) comprises a step of vacuum drying said brownish or dark red liquid to form said crude product.

28. The method, as recited in claim 14, further comprising the steps of:
(g) dissolving said crude product by adding water to form a solution; and
(h) removing precipitates of said solution and forming a raw product.

29. The method, as recited in claim 28, wherein the step (h) includes the steps of centrifuging said solution to remove said precipitates and concentrating and drying said solution after removing said precipitates to obtain said raw product.

30. The method, as recited in claim 28, further comprising the steps of:
(i) dividing said raw product into a first portion in brownish black color, a second portion in brown color and a third portion in yellow color; and
(j) concentrating and drying said first, second and third portions of said raw product.

31. The method, as recited in claim 30, wherein in the step (i), said raw product is divided into said first, second and third portion by sephadex LH 20 and water.

32. The method, as recited in claim 29, further comprising the steps of:
(i) dividing said raw product into a first portion in brownish black color, a second portion in brown color and a third portion in yellow color; and
(j) concentrating and drying said first, second and third portions of said raw product.

33. The method, as recited in claim 32, wherein in the step (i), said raw product is divided into said first, second and third portion by sephadex LH 20 and water.

34. The method, as recited in claim 15, further comprising the steps of:
(g) dissolving said crude product by adding water to form a solution; and
(h) removing precipitates of said solution and forming a raw product.

35. The method, as recited in claim 34, wherein the step (h) includes the steps of centrifuging said solution to remove said precipitates and concentrating and drying said solution after removing said precipitates to obtain said raw product.

36. The method, as recited in claim 34, further comprising the steps of:
(i) dividing said raw product into a first portion in brownish black color, a second portion in brown color and a third portion in yellow color; and
(j) concentrating and drying said first, second and third portions of said raw product.

37. The method, as recited in claim 36, wherein in the step (i), said raw product is divided into said first, second and third portion by sephadex LH 20 and water.

38. The method, as recited in claim 35, further comprising the steps of:
(i) dividing said raw product into a first portion in brownish black color, a second portion in brown color and a third portion in yellow color; and
(j) concentrating and drying said first, second and third portions of said raw product.

39. The method, as recited in claim 38, wherein in the step (i), said raw product is divided into said first, second and third portion by sephadex LH 20 and water.

40. The method, as recited in claim 27, further comprising the steps of:
(g) dissolving said crude product by adding water to form a solution; and
(h) removing precipitates of said solution and forming a raw product.

41. The method, as recited in claim 40, wherein the step (h) includes the steps of centrifiging said solution to remove said precipitates and concentrating and drying said solution after removing said precipitates to obtain said raw product.

42. The method, as recited in claim 40, further comprising the steps of:
(i) dividing said raw product into a first portion in brownish black color, a second portion in brown color and a third portion in yellow color; and
(j) concentrating and drying said first, second and third portions of said raw product.

43. The method, as recited in claim 42, wherein in the step (i), said raw product is divided into said first, second and third portion by sephadex LH 20 and water.

44. The method, as recited in claim 41, further comprising the steps of:
(i) dividing said raw product into a first portion in brownish black color, a second portion in brown color and a third portion in yellow color; and
(j) concentrating and drying said first, second and third portions of said raw product.

45. The method, as recited in claim 44, wherein in the step (i), said raw product is divided into said first, second and third portion by sephadex LH 20 and water.

46. The method, as recited in claim 15, wherein the step (a) comprises the steps of cleaning, powdering, centrifuging, filtering, and extracting a 20 kg of said Stellaria plant to get said waster extraction,
wherein the step (b) comprises a step of precipitating and centrifuging said water extraction to get said liquid,
wherein the step (c) comprises a step of passing said liquid through said macroporous resin column (id 10 cm×120 cm) with a flow speed from 1 ml per minute to 3 ml per minute,
wherein the step (d) comprises the steps of discarding a flowing portion of said liquid and washing another remaining portion of said liquid of said macroporous resin column by running water thoroughly,
wherein the step (e) comprises the steps of washing said macroporous resin column with said 5% ethanol for a volume twice as much as a column volume of said macroporous resin column to get yellow color liquid, discarded, and eluting and collecting said brownish or dark red liquid by said 60% ethanol for a volume quintuple as much as said column volume,
wherein the step (f) comprises the steps of concentrating said brownish or dark red liquid by collecting said ethanol therein by means of a pressure-reduction dryer to form a concentrated viscous liquid, about 100 ml, centrifuging said concentrated viscous liquid in a speed of 4000 r per minute for 5 minutes to remove said precipitates to form a pre-crude product while residues are discarded, and drying said pre-crude product to form said crude product.

47. The method, as recited in claim 45, wherein the step (a) comprises the steps of cleaning, powdering, centrifuging, filtering, and extracting a 20 kg of said Stellaria plant to get said waster extraction,
wherein the step (b) comprises the steps of precipitating and centrifuging said water extraction to get said liquid,
wherein the step (c) comprises the steps of passing said liquid through said macroporous resin column (id 10 cm×120 cm) with a flow speed from 1 ml per minute to 3 ml per minute,
wherein the step (d) comprises the steps of discarding a flowing portion of said liquid and washing another remaining portion of said liquid of said macroporous resin column by running water thoroughly,
wherein the step (e) comprises the steps of washing said macroporous resin column with said 5% ethanol for a volume twice as much as a column volume of said macroporous resin column to get yellow color liquid, discarded, and eluting and collecting said brownish or dark red liquid by said 60% ethanol for a triple as much as said column volume,
wherein the step (f) comprises the steps of concentrating said brownish or dark red liquid by collecting said ethanol therein by means of a pressure-reduction dryer to form a concentrated viscous liquid, about 100 ml, centrifuging said concentrated viscous liquid in a speed of 4000 r per minute for 5 minutes to remove said precipitates to form a pre-crude product while residues are discarded, and drying said pre-crude product to form said crude product,
wherein the step (g) comprises a step of adding 200 ml water to said crude product to form said solution,
wherein the step (h) comprises the steps of centrifuging said solution in a speed of 4000 r per minute for 5 minutes to remove said precipitates to obtain a supernatant, filtering said supernatant is filtered through a 0.22 μm filter membrane in a bacteria free container, concentrating said solution under a reduced pressure to form an ointment, and drying said ointment to obtain said raw product which has a dark brownish red color,
wherein the step (i) comprises the steps of adding 50 ml water to 100 g of said raw product to form said solution, adding said solution on top of said sephadex LH20 column, eluting and collecting said first portion of said raw product by using water double as much as a column volume of said sephadex LH20 column, eluting and collecting said second portion of said raw product by using water double as much as said column volume of said sephadex LH20 column, and eluting and collecting said third portion of said raw product by using 95% ethanol double as much as said column volume of said sephadex LH20 column, wherein solutions of said first, second and third portions of said raw product are concentrated and dried respectively to form 15% of said first portion, 67% of said second portion and 15% of said third portion of said raw product.

48. The method, as recited in claim 14, wherein said agent includes a sodium chloride solution.

49. The method, as recited in claim 14, wherein said resin column is an anion exchanging resin column.

50. The method, as recited in claim 48, wherein said resin column is an anion exchanging resin column.

51. The method, as recited in claim 50, wherein the step (b) comprises a step of precipitating and centrifuging said water extraction to obtain said liquid.

52. The method, as recited in claim 50, wherein the step (b) comprises a step of filtering said water extraction to obtain said liquid.

53. The method, as recited in claim 50, wherein the step (c) further comprises a step of absorbing condensate compounds of said total organic and phenolic acid glycosides and salts of total organic and phenolic acid glycosides of said liquid.

54. The method, as recited in claim 52, wherein the step (c) further comprises a step of absorbing condensate compounds of said total organic and phenolic acid glycosides and salts of total organic and phenolic acid glycosides of said liquid.

55. The method, as recited in claim 50, wherein the step (f) comprises the steps of desalinating, concentrating and filtering effective materials to form said crude product.

56. The method, as recited in claim 54, wherein the step (f) comprises the steps of desalinating, concentrating and filtering effective materials to form said crude product.

57. The method, as recited in claim 50, further comprising the steps of:
(g) dissolving said crude product by adding water to form a solution; and
(h) removing precipitates of said solution and forming a raw product.

58. The method, as recited in claim 57, wherein the step (h) includes the steps of centrifuging said solution to remove said precipitates and concentrating and drying said solution after removing said precipitates to obtain said raw product.

59. The method, as recited in claim 57, further comprising the steps of:
(i) dividing said raw product into a first portion having a brownish black color, a second portion having a brown color and a third portion having a yellow color.

60. The method, as recited in claim 59, wherein said first, second and third portions of said raw product are divided by a sephadex LH20 column and water.

61. The method, as recited in claim 58, further comprising the steps of:
(i) dividing said raw product into a first portion having a brownish black color, a second portion having a brown color and a third portion having a yellow color by a sephadex LH20 column and water.

62. The method, as recited in claim 56, further comprising the steps of:
(g) dissolving said crude product by adding water to form a solution; and
(h) removing precipitates of said solution and forming a raw product.

63. The method, as recited in claim 62, wherein the step (h) includes the steps of centrifuging said solution to remove said precipitates and concentrating and drying said solution after removing said precipitates to obtain said raw product.

64. The method, as recited in claim 62, further comprising the steps of:
(i) dividing said raw product into a first portion having a brownish black color, a second portion having a brown color and a third portion having a yellow color.

65. The method, as recited in claim 64, wherein said first, second and third portions of said raw product are divided by a sephadex LH20 column and water.

66. The method, as recited in claim 63, further comprising the steps of:
(i) dividing said raw product into a first portion having a brownish black color, a second portion having a brown color and a third portion having a yellow color by a sephadex LH20 column and water.

67. The method, as recited in claim 56, wherein the step (a) comprises the steps of cleaning, powdering, centrifuging, filtering, and extracting a 2.5 kg of said Stellaria plant to get said waster extraction,
wherein the step (b) comprises a step of precipitating and centrifuging said water extraction to get said liquid,
wherein the step (c) comprises a step of passing said liquid through said anion exchanging resin column (id 10 cm×120 cm),
wherein the step (d) comprises a step of cleaning said ion exchanging column by adding said selected from a group consisting of distilled water and de-ionized water for a volume triple as much as a column volume of said ion exchanging column,
wherein the step (e) comprises a step of cleaning said ion exchanging column by adding said sodium chloride solution for a volume twice as much as said column volume to get said brownish or dark red color liquid,
wherein the step (f) comprises the steps of concentrating said brownish or dark red liquid by collecting said sodium chloride solution therein by means of a pressure-reduction and semi-permeable membrane package until said solution is dialysized with running water to an extent being tested inactive with AgNO3 to form a concentrated viscous liquid, and drying said concentrated viscous liquid to form said crude product.

68. The method, as recited in claim 60, wherein the step (a) comprises the steps of cleaning, powdering, centrifuging, filtering, and extracting a 2.5 kg of said Stellaria plant to get said waster extraction,
wherein the step (b) comprises a step of precipitating and centrifuging said water extraction to get said liquid,
wherein the step (c) comprises a step of passing said liquid through said anion exchanging resin column (id 10 cm×120 cm),
wherein the step (d) comprises a step of cleaning said ion exchanging column by adding said selected from a group consisting of distilled water and de-ionized water for a volume triple as much as a column volume of said ion exchanging column,
wherein the step (e) comprises a step of cleaning said ion exchanging column by adding said sodium chloride solution for a volume twice as much as said column volume to get said brownish or dark red color liquid,
wherein the step (f) comprises the steps of concentrating said brownish or dark red liquid by collecting said sodium chloride solution therein by means of a pressure-reduction and semi-permeable membrane package until said solution is dialysized with running water to an extent being tested inactive with AgNO3 to form a concentrated viscous liquid, and drying said concentrated viscous liquid to form said crude product,
wherein the step (g) comprises a step of adding 250 ml water to said crude product to form said solution;
wherein the step (h) comprises the steps of centrifuging said solution in a speed of 4000 r per minute for 5 minutes to remove said precipitates to obtain a supernatant, filtering said supernatant through a 0.22 μm filter membrane in a bacteria free container, concentrating said solution under a reduced pressure to form ointment, and drying said ointment to obtain said raw product having a dark brownish red color,
wherein the step (i) comprises the steps of adding 50 ml water to 100 g of said raw product to form said solution, adding said solution to a top of said sephadex LH 20 column, eluting and collecting said first portion of said raw product by using water double as much as a column volume of said sephadex LH20 column, eluting and collecting said second portion of said raw product by using water double as much as said column volume of said sephadex LH20 column, and eluting and collecting said third portion of said third portion of said raw product by using 95% ethanol double as much as said column volume of said sephadex LH20 column, wherein solutions of said first, second and third portions of said raw product are concentrated and dried respectively to form 15% of said first portion, 67% of said second portion and 15% of said third portion of said raw product.

69. The method, as recited in claim 66, wherein the step (a) comprises the steps of cleaning, powdering, centrifuging, filtering, and extracting a 2.5 kg of said Stellaria plant to get said waster extraction,
wherein the step (b) comprises a step of precipitating and centrifuging said water extraction to get said liquid,
wherein the step (c) comprises a step of passing said liquid through said anion exchanging resin column (id 10 cm×120 cm),
wherein the step (d) comprises a step of cleaning said ion exchanging column by adding said selected from a group consisting of distilled water and de-ionized water for a volume triple as much as a column volume of said ion exchanging column,
wherein the step (e) comprises a step of cleaning said ion exchanging column by adding said sodium chloride solution for a volume twice as much as said column volume to get said brownish or dark red color liquid,
wherein the step (f) comprises the steps of concentrating said brownish or dark red liquid by collecting said sodium chloride solution therein by means of a pressure-reduction and semi-permeable membrane package until said solution is dialysized with running water to an extent being tested inactive with AgNO3 to form a concentrated viscous liquid, and drying said concentrated viscous liquid to form said crude product,
wherein the step (g) comprises a step of adding 250 ml water to said crude product to form said solution;
wherein the step (h) comprises the steps of centrifuging said solution in a speed of 4000 r per minute for 5 minutes to remove said precipitates to obtain a supernatant, filtering said supernatant through a 0.22 μm filter membrane in a bacteria free container, concentrating said solution under a reduced pressure to form ointment, and drying said ointment to obtain said raw product having a dark brownish red color,
wherein the step (i) comprises the steps of adding 50 ml water to 100 g of said raw product to form said solution, adding said solution to a top of said sephadex LH 20 column, eluting and collecting said first portion of said raw product by using water double as much as a column volume of said sephadex LH20 column, eluting and collecting said second portion of said raw product by using water double as much as said column volume of said sephadex LH20 column, and eluting and collecting said third portion of said third portion of said raw product by using 95% ethanol double as much as said column volume of said sephadex LH20 column, wherein solutions of said first, second and third portions of said raw product are concentrated and dried respectively to form 15% of said first portion, 67% of said second portion and 15% of said third portion of said raw product.
